(19) United States
(12) Patent Application Publication
(10) Pub. No.: US 2026/0317973 A1
(43) Pub. Date: Oct. 15, 2026

(54) A Forming Surface for Nonwoven Material and Methods of Manufacture

(71) Applicant: Kimberly-Clark Worldwide, Inc., Neenah, WI (US)

(72) Inventors: Antonio J. Carrillo Ojeda, Appleton, WI (US); Steven J. Roffers, Neenah, WI (US); Dustin J. Smith, Green Bay, WI (US); Folau Piula Penaia, Altona, Victoria (AU); Adam J. Kurzynski, Neenah, WI (US); James L. Cahall, Appleton, WI (US); Teresa Kubicki, Greensborough, Victoria (AU)

(21) Appl. No.: 19/108,537

(22) PCT Filed: Sep. 21, 2022
(86) PCT No.: PCT/US2022/044258

§ 371 Date: Mar. 4, 2025

(51) Int. Cl. D04H 1/732

(52) U.S. Cl. CPC D04H 1/732; D10B 2509/026

(57) ABSTRACT

A forming surface for fluid-entangled nonwoven material includes a wall with a plurality of formation holes. Each of the plurality of formation holes includes a blind hole and at least one outlet passage. An end portion of each blind hole is recessed within the wall.

### BACKGROUND

Nonwoven materials are frequently used within personal care absorbent articles, such as diapers or incontinence briefs. Moreover, an inner lining of such articles face and contact the skin of a wearer. Contact between body exudates, such as semi solid fecal material, captured within the articles and the skin of the wearer can cause discomfort. Moving body exudates through the inner lining and away from the skin of the wearer can reduce or limit such discomfort. Thus, a nonwoven material with features that facilitate movement of body exudates through the nonwoven material would be useful.

Nonwoven materials with three-dimensional topography can facilitate movement of body exudates through the nonwoven material; however, known methods and mechanisms for forming nonwoven materials with three-dimensional topography suffer drawbacks, including reduced material thickness, material stiffness, and low resiliency. Thus, a nonwoven material with improved three-dimensional topography would be useful.

### SUMMARY

In general, the present disclosure is directed to a forming surface for fluid-entangled nonwoven material. The forming surface may include blind or socketed formation holes that are configured for supporting distal ends of nodes during the fluid-entangling process to form the nodes with desirable material distribution, which can advantageously increase a compression linearity of the nodes relative to conventional nodes and/or may increase an anisotropy value of the nodes relative to conventional nodes. The nonwoven material with the nodes may be incorporated within an absorbent article, such as a pad, diaper, disposable undergarment, etc.

In one example embodiment, a forming surface for fluid-entangled nonwoven material includes a wall with a first surface and a second surface. The first surface is positioned opposite the second surface on the wall. The wall defines a plurality of formation holes. Each of the plurality of formation holes includes a blind hole and at least one outlet passage. Each blind hole extends from a first end portion to a second end portion. The first end portion of each blind hole is positioned at the first surface of the wall. Each blind hole extends towards the second surface of the wall such that the second end portion of each blind hole is recessed within the wall. Each of the at least one outlet passage extends from a first end portion to a second end portion. The first end portion of each of the at least one outlet passage is positioned at the second end portion of each blind hole. The second end portion of each of the at least one outlet passage is positioned at the second surface of the wall. For each of the plurality of formation holes, an area of the blind hole at the first end portion of the blind hole is greater than a collective area of the at least one outlet passage at the first end portion of the at least one passage. A ratio of the area of the blind hole at the first end portion of the blind hole to the collective area of the at least one outlet passage at the first end portion of the at least one passage is between 2.75:1 and 11:1.

In another example embodiment, a forming surface for fluid-entangled nonwoven material includes a wall with a first surface and a second surface. The first surface is positioned opposite the second surface on the wall. The wall defines a plurality of formation holes. Each of the plurality of formation holes includes a blind hole and at least one outlet passage. Each blind hole extends from a first end portion to a second end portion. The first end portion of each blind hole is positioned at the first surface of the wall. Each blind hole extends towards the second surface of the wall such that the second end portion of each blind hole is recessed within the wall. Each of the at least one outlet passage extends from a first end portion to a second end portion. The first end portion of each of the at least one outlet passage is positioned at the second end portion of each blind hole. The second end portion of each of the at least one outlet passage is positioned at the second surface of the wall. The first and second end portions of each blind hole are spaced apart along a central axis of each blind hole. The first end portion of each of the at least one outlet passage is offset from the central axis of each blind hole.

In another example embodiment, a forming surface for fluid-entangled nonwoven material includes a wall having a first surface and a second surface. The first surface is positioned opposite the second surface on the wall. The wall defines a plurality of formation holes. Each of the plurality of formation holes includes a blind hole and at least one outlet passage. Each blind hole extends from a first end portion to a second end portion. The first end portion of each blind hole is positioned at the first surface of the wall. Each blind hole extending towards the second surface of the wall such that the second end portion of each blind hole is recessed within the wall. The wall includes a connecting surface and a formation surface. The connecting surface extends inwardly into the blind hole from the first end portion of the blind hole. A cross-section of the connecting surface in a plane perpendicular to a central axis of the blind hole is substantially constant along the central axis. The formation surface is positioned proximate the second end portion of the blind hole. A cross-section of the formation surface in the plane perpendicular to the central axis of the blind hole decreases along the central axis towards the second end portion of the blind hole.

These and other features, aspects and advantages of the present disclosure will become better understood with reference to the following description and appended claims. The accompanying drawings, which are incorporated in and constitute a part of this specification, illustrate embodiments of the disclosure and, together with the description, serve to explain the principles of the disclosure.

### BRIEF DESCRIPTION OF THE DRAWINGS

A full and enabling disclosure of the present disclosure, including the best mode thereof, directed to one of ordinary skill in the art, is set forth in the specification, which makes reference to the appended figures.

FIG. 1 is a top, plan view of an absorbent article according to an example embodiment of the present disclosure and in a stretched, laid flat, unfastened condition.

FIG. 2 is a top, plan view of a nonwoven material according to an example embodiment of the present disclosure.

FIG. 3 is an image providing a detailed view of nodes of the example nonwoven material of FIG. 1.

FIG. 4 is a partial side, section view of the example nonwoven material of FIG. 2 taken along line 4-4.

FIG. 5 is a schematic side view of an apparatus and process for manufacturing a nonwoven material according to an example embodiment of the present disclosure.

FIG. 6 is a schematic side view of an apparatus and process for manufacturing a nonwoven material according to another example embodiment of the present disclosure.

FIG. 7 is a schematic side view of an apparatus and process for manufacturing a nonwoven material according to yet another example embodiment of the present disclosure.

FIG. 8 is a schematic side view of an apparatus and process for manufacturing a nonwoven material according to a further example embodiment of the present disclosure.

FIG. 9 is a side section view of a formation surface according to an example embodiment of the present disclosure.

FIG. 10 is a side section view of a formation hole of the example formation surface of FIG. 9.

FIG. 11 is a side section view of the formation hole of FIG. 10 during a fluid-entangled operation on a nonwoven web.

FIG. 12 is a side section view of a formation hole according to another example embodiment of the present subject matter.

FIG. 13 is a side section view of a formation hole according to another example embodiment of the present subject matter.

FIG. 14 is a side section view of a formation hole according to another example embodiment of the present subject matter.

FIG. 15 is a side section view of a formation hole according to another example embodiment of the present subject matter.

FIG. 16 is a perspective view of example equipment and set-up to perform the Material Sample Analysis Test Method as described herein.

Repeat use of reference characters in the present specification and drawings is intended to represent the same or analogous features or elements of the present invention.

### DETAILED DESCRIPTION

In an embodiment, the present disclosure is generally directed towards a forming surface, which can be used to form fluid-entangled nonwoven material with improved three-dimensional topography. It is to be understood by one of ordinary skill in the art that the present discussion is a description of exemplary embodiments only, and is not intended as limiting the broader aspects of the present disclosure.

When introducing elements of the present disclosure or the preferred embodiment(s) thereof, the articles “a”, “an”, “the” and “said” are intended to mean that there are one or more of the elements. As used herein, the terms “includes” and “including” are intended to be inclusive in a manner similar to the term “comprising.” Similarly, the term “or” is generally intended to be inclusive (i.e., “A or B” is intended to mean “A or B or both”). Approximating language, as used herein throughout the specification and claims, is applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. For example, the approximating language may refer to being within a ten percent (10%) margin.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “fluid entangling”, and “fluid-entangled” generally refers herein to a formation process for further increasing the degree of fiber entanglement within a given fibrous nonwoven web or between fibrous nonwoven webs and other materials so as to make the separation of the individual fibers and/or the layers more difficult as a result of the entanglement. Generally, this is accomplished by supporting the fibrous nonwoven web on some type of forming or carrier surface which has at least some degree of permeability to the impinging pressurized fluid. A pressurized fluid stream (usually multiple streams) is then directed against the surface of the nonwoven web which is opposite the supported surface of the web. The pressurized fluid contacts the fibers and forces portions of the fibers in the direction of the fluid flow thus displacing all or a portion of a plurality of the fibers towards the supported surface of the web. The result is a further entanglement of the fibers in what can be termed the Z-direction of the web (the thickness) relative to its more planar dimension, the X-Y plane. When two or more separate webs or other layers are placed adjacent one another on the forming/carrier surface and subjected to the pressurized fluid, the generally desired result is that some of the fibers of at least one of the webs are forced into the adjacent web or layer thereby causing fiber entanglement between the interfaces of the two surfaces so as to result in the bonding or joining of the webs/layers together due to the increased entanglement of the fibers. The degree of bonding or entanglement will depend on a number of factors including, but not limited to, the types of fibers being used, the fiber lengths, the degree of pre-bonding or entanglement of the web or webs prior to subjection to the fluid entangling process, the type of fluid being used (liquids, such as water, steam or gases, such as air), the pressure of the fluid, the number of fluid streams, the speed of the process, the dwell time of the fluid and the porosity of the web or webs/other layers and the forming/carrier surface. One of the most common fluid entangling processes is referred to as hydroentangling, which is a well-known process to those of ordinary skill in the art of nonwoven webs. Examples of fluid entangling process can be found in U.S. Pat. No. 4,939,016 to Radwanski et al., U.S. Pat. No. 3,485,706 to Evans, and U.S. Pat. Nos. 4,970,104 and 4,959,531 to Radwanski, each of which is incorporated herein in its entirety by reference thereto for all purposes.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, reductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, diaper pant, training pant, youth pant, incontinent product, or other absorbent article about the wearer of one of these absorbent articles. A user and a wearer can be one and the same person.

### Absorbent Article:

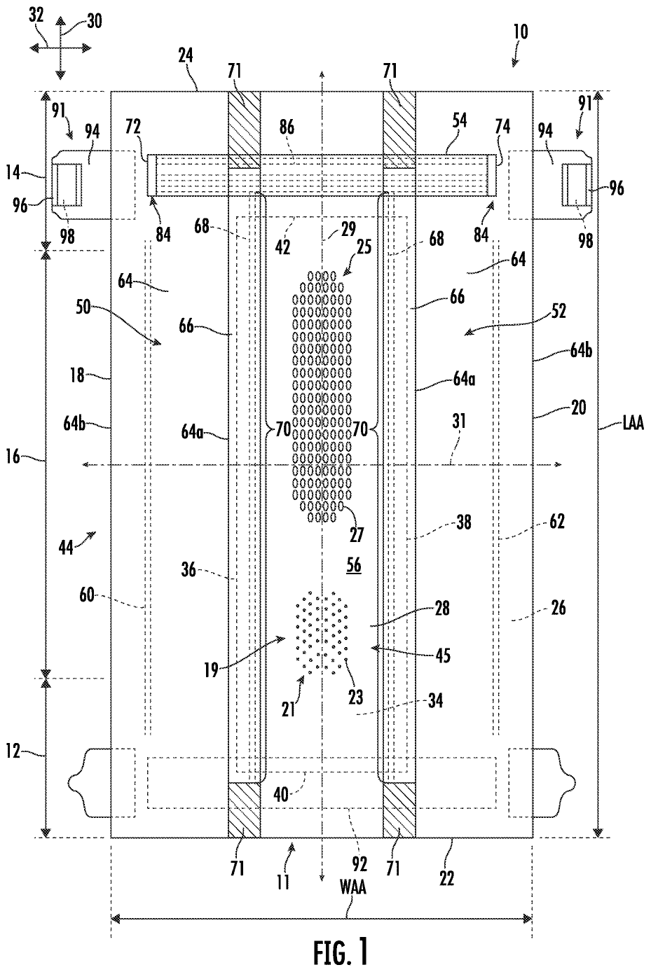

FIG. 1 is a top, plan view of an absorbent article 10 according to an example embodiment of the present disclosure and in a stretched, laid flat, unfastened condition. While absorbent article 10 is shown as a diaper in the example embodiment shown in FIG. 1, it will be understood that absorbent article 10 may be configured as other types of absorbent articles, such as training pants, youth pants, adult incontinence garments, and feminine hygiene articles, and the like, in other example embodiments. While the example embodiments and illustrations described herein may generally apply to absorbent articles manufactured in the product longitudinal direction, which is hereinafter called the machine direction or process direction manufacturing of a product, it should be noted that one of ordinary skill in the art could apply the information herein to absorbent articles manufactured in the latitudinal direction of the product, which hereinafter is called the cross direction manufacturing of a product, without departing from the spirit and scope of the disclosure.

The absorbent article 10 illustrated in FIG. 1 may include a chassis 11. The absorbent article 10 may also include a front waist region 12, a rear waist region 14, and a crotch region 16 disposed between the front waist region 12 and the rear waist region 14 and interconnecting the front and rear waist regions, 12, 14, respectively. The front waist region 12 may be referred to as the front end region, the rear waist region 14 may be referred to as the rear end region, and the crotch region 16 may be referred to as the intermediate region. With respect to an article manufactured in a cross-direction manufacturing process, for example in a three-piece construction, such an absorbent article may have a chassis including a front waist panel defining the front waist region, a rear waist panel defining the rear waist region, and an absorbent panel defining the crotch region. The absorbent panel may extend between the front waist panel and the rear waist panel. In some example embodiments, the absorbent panel may overlap the front waist panel and the rear waist panel. The absorbent panel may be bonded to the front waist panel and the rear waist panel to define a three-piece construction. However, it is contemplated that an absorbent article may be manufactured in a cross-direction without being a three-piece construction garment.

The absorbent article 10 may have a pair of longitudinal side edges 18, 20, and a pair of opposite waist edges, respectively designated front waist edge 22 and rear waist edge 24. The front waist region 12 may be contiguous with the front waist edge 22 and the rear waist region 14 may be contiguous with the rear waist edge 24. The longitudinal side edges 18, 20 may extend from the front waist edge 22 to the rear waist edge 24. The longitudinal side edges 18, 20 may extend in a direction parallel to the longitudinal direction 30 for their entire length, such as for the absorbent article 10. In other example embodiments, the longitudinal side edges 18, 20 may be curved between the front waist edge 22 and the rear waist edge 24.

The front waist region 12 may include the portion of the absorbent article 10 that, when worn, is positioned at least in part on the front of the wearer while the rear waist region 14 may include the portion of the absorbent article 10 that, when worn, is positioned at least in part on the back of the wearer. The crotch region 16 of the absorbent article 10 may include the portion of the absorbent article 10 that, when worn, is positioned between the legs of the wearer and may partially cover the lower torso of the wearer. The waist edges, 22 and 24, of the absorbent article 10 may be configured to encircle the waist of the wearer and together define a central waist opening for the waist of the wearer. Portions of the longitudinal side edges 18, 20 in the crotch region 16 may generally define leg openings for the legs of the wearer when the absorbent article 10 is worn.

The absorbent article 10 may include an outer cover 26 and a bodyside liner 28. The outer cover 26 and the bodyside liner 28 may form a portion of the chassis 11. In an example embodiment, the bodyside liner 28 may be bonded to the outer cover 26 in a superposed relation by any suitable mechanism such as, but not limited to, adhesives, ultrasonic bonds, thermal bonds, pressure bonds, or other conventional techniques. The outer cover 26 may define a length in a longitudinal direction 30, and a width in the lateral direction 32, which, in the illustrated example embodiment, may coincide with the length LAA and width WAA of the absorbent article 10. As illustrated in FIG. 1, the absorbent article 10 may have a longitudinal axis 29 extending in the longitudinal direction 30 and a lateral axis 31 extending in the lateral direction 32.

The chassis 11 may include an absorbent body 34. The absorbent body 34 may be disposed between the outer cover 26 and the bodyside liner 28. The absorbent body 34 may have longitudinal edges, 36 and 38, which, in an example embodiment, may form portions of the longitudinal side edges, 18 and 20, respectively, of the absorbent article 10. The absorbent body 34 may have a first end edge 40 that is opposite a second end edge 42, respectively, which, in an example embodiment, may form portions of the waist edges, 22 and 24, respectively, of the absorbent article 10. In some example embodiments, the first end edge 40 may be in the front waist region 12. In some example embodiments, the second end edge 42 may be in the rear waist region 14. In an example embodiment, the absorbent body 34 may have a length and width that are the same as or less than the length LAA and width WAA of the absorbent article 10. The bodyside liner 28, the outer cover 26, and the absorbent body 34 may form part of an absorbent assembly 44. In example embodiments of articles according to aspects of the present disclosure, which are manufactured in a cross-direction manufacturing process, the absorbent body 34 may form the absorbent assembly 44. The absorbent assembly 44 may also include a fluid transfer layer (not shown) and/or a fluid acquisition layer (not shown) between the bodyside liner 28 and the absorbent body 34 as is known in the art. The absorbent assembly 44 may also include a spacer layer (not shown) disposed between the absorbent body 34 and the outer cover 26.

The absorbent article 10 may be configured to contain and/or absorb liquid, solid, and semi-solid body exudates discharged from the wearer. In some example embodiments, containment flaps 50, 52 may be configured to provide a barrier to the lateral flow of body exudates. To further enhance containment and/or absorption of body exudates, the absorbent article 10 may suitably include an elasticated waist member 54. In some example embodiments, the elasticated waist member 54 may be disposed in the rear waist region 14 of the absorbent article 10. Although, it is contemplated that the elasticated waist member 54 may be additionally or alternatively disposed in the front waist region 12 of the absorbent article 10.

The elasticated waist member 54 may be disposed on the body facing surface 19 of the chassis 11 to help contain and/or absorb body exudates. In some example embodiments, such as in the absorbent article 10 depicted in FIG. 1, the elasticated waist member 54 may be disposed on the body facing surface 45 of the absorbent assembly 44. In some example embodiments, the elasticated waist member 54 may be disposed at least partially on the body facing surface 56 of the bodyside liner 28.

The absorbent article 10 may further include leg elastic members 60, 62 as are known to those skilled in the art. The leg elastic members 60, 62 may be attached to the outer cover 26 and/or the bodyside liner 28 along the opposite longitudinal side edges, 18 and 20, and positioned in the crotch region 16 of the absorbent article 10. The leg elastic members 60, 62 may be parallel to the longitudinal axis 29 as shown in FIG. 1, or may be curved as is known in the art. The leg elastic members 60, 62 may provide elasticized leg cuffs.

The outer cover 26 and/or portions thereof may be breathable and/or liquid impermeable. The outer cover 26 and/or portions thereof may be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an example embodiment, for example, the outer cover 26 may be constructed of a microporous polymeric film, such as polyethylene or polypropylene.

In an example embodiment, the outer cover 26 may be a single layer of a liquid impermeable material, such as a polymeric film. In an example embodiment, the outer cover 26 may be suitably stretchable, and more suitably elastic, in at least the lateral direction 32 of the absorbent article 10. In an example embodiment, the outer cover 26 may be stretchable, and more suitably elastic, in both the lateral 32 and the longitudinal 30 directions. In an example embodiment, the outer cover 26 may be a multi-layered laminate in which at least one of the layers is liquid impermeable. In some example embodiments, the outer cover 26 may be a two-layer construction, including an outer layer (not shown) and an inner layer (not shown) which may be bonded together such as by a laminate adhesive. Suitable laminate adhesives may be applied continuously or intermittently as beads, a spray, parallel swirls, or the like, but it is to be understood that the inner layer may be bonded to the outer layer by other bonding methods, including, but not limited to, ultrasonic bonds, thermal bonds, pressure bonds, or the like.

The outer layer of the outer cover 26 may be any suitable material and may be one that provides a generally cloth-like texture or appearance to the wearer. An example of such material may be a 100% polypropylene bonded-carded web with a diamond bond pattern available from Sandler A.G., Germany, such as 30 gsm Sawabond 4185® or equivalent. Another example of material suitable for use as an outer layer of an outer cover 26 may be a 20 gsm spunbond polypropylene non-woven web. The outer layer may also be constructed of the same materials from which the bodyside liner 28 may be constructed as described herein.

The liquid impermeable inner layer of the outer cover 26 (or the liquid impermeable outer cover 26 where the outer cover 26 is of a single-layer construction) may be either vapor permeable (i.e., “breathable”) or vapor impermeable. The liquid impermeable inner layer (or the liquid impermeable outer cover 26 where the outer cover 26 is of a single-layer construction) may be manufactured from a thin plastic film. The liquid impermeable inner layer (or the liquid impermeable outer cover 26 where the outer cover 26 is of a single-layer construction) may inhibit liquid body exudates from leaking out of the absorbent article 10 and wetting articles, such as bed sheets and clothing, as well as the wearer and caregiver.

In some example embodiments, where the outer cover 26 is of a single layer construction, it may be embossed and/or matte finished to provide a more cloth-like texture or appearance. The outer cover 26 may permit vapors to escape from the absorbent article 10 while preventing liquids from passing through. A suitable liquid impermeable, vapor permeable material may be composed of a microporous polymer film or a non-woven material which has been coated or otherwise treated to impart a desired level of liquid impermeability.

The absorbent body 34 may be suitably constructed to be generally compressible, conformable, pliable, non-irritating to the wearer's skin and capable of absorbing and retaining liquid body exudates. The absorbent body 34 may be manufactured in a wide variety of sizes and shapes (for example, rectangular, trapezoidal, T-shape, I-shape, hourglass shape, etc.) and from a wide variety of materials. The size and the absorbent capacity of the absorbent body 34 should be compatible with the size of the intended wearer (infants to adults) and the liquid loading imparted by the intended use of the absorbent article 10. The absorbent body 34 may have a length and width that may be less than or equal to the length LAA and width WAA of the absorbent article 10.

In an example embodiment, the absorbent body 34 may be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an example embodiment, the absorbent body 34 may be a matrix of cellulosic fluff and superabsorbent material. In further example embodiments, the absorbent body 34 may comprise mostly superabsorbent material, or even greater than 80% superabsorbent material, greater than 90% superabsorbent material, or comprise 100% superabsorbent material, by weight of absorbent material of the absorbent body 34. Although, in other example embodiments, the absorbent body 34 may be free of superabsorbent material. In an example embodiment, the absorbent body 34 may be constructed of a single layer of materials, or in the alternative, may be constructed of two or more layers of materials.

Various types of wettable, hydrophilic fibers may be used in the absorbent body 34. Examples of suitable fibers include: natural fibers; cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. Suitable superabsorbent materials may be selected from natural, synthetic, and modified natural polymers and materials. The superabsorbent materials may be inorganic materials, such as silica gels, or organic compounds, such as cross-linked polymers.

If a spacer layer is present, the absorbent body 34 may be disposed on the spacer layer and superposed over the outer cover 26. The spacer layer may be bonded to the outer cover 26, for example, by adhesive. In some example embodiments, a spacer layer may not be present and the absorbent body 34 may directly contact the outer cover 26 and may be directly bonded to the outer cover 26. However, it is to be understood that the absorbent body 34 may be in contact with, and not bonded with, the outer cover 26 and remain within the scope of this disclosure. In an example embodiment, the outer cover 26 may be composed of a single layer and the absorbent body 34 may be in contact with the singer layer of the outer cover 26. In some example embodiments, at least a portion of a layer, such as but not limited to, a fluid transfer layer and/or a spacer layer, may be positioned between the absorbent body 34 and the outer cover 26. The absorbent body 34 may be bonded to the fluid transfer layer and/or the spacer layer.

The bodyside liner 28 of the absorbent article 10 may overlay the absorbent body 34 and the outer cover 26 and may isolate the wearer's skin from liquid waste retained by the absorbent body 34. In various example embodiments, a fluid transfer layer may be positioned between the bodyside liner 28 and the absorbent body 34. In various example embodiments, an acquisition layer (not shown) may be positioned between the bodyside liner 28 and the absorbent body 34 or a fluid transfer layer, if present. In various example embodiments, the bodyside liner 28 may be bonded to the acquisition layer, or to the fluid transfer layer if no acquisition layer is present, via adhesive and/or by a point fusion bonding. The point fusion bonding may be selected from ultrasonic, thermal, pressure bonding, and combinations thereof.

In an example embodiment, the bodyside liner 28 may extend beyond the absorbent body 34 and/or a fluid transfer layer, if present, and/or an acquisition layer, if present, and/or a spacer layer, if present, to overlay a portion of the outer cover 26 and may be bonded thereto by any method deemed suitable, such as, for example, by being bonded thereto by adhesive, to substantially enclose the absorbent body 34 between the outer cover 26 and the bodyside liner 28. It is contemplated that the bodyside liner 28 may be narrower than the outer cover 26. However, in other example embodiments, the bodyside liner 28 and the outer cover 26 may be of the same dimensions in width and length, for example, as may be seen in the example embodiments illustrated in FIG. 1. In other example embodiments, the bodyside liner 28 may be of greater width than the outer cover 26. It is also contemplated that the bodyside liner 28 may not extend beyond the absorbent body 34 and/or may not be secured to the outer cover 26. In some example embodiments, the bodyside liner 28 may wrap at least a portion of the absorbent body 34, including wrapping around both longitudinal edges 36, 38 of the absorbent body 34, and/or one or more of the end edges 40, 42. It is further contemplated that the bodyside liner 28 may be composed of more than one segment of material. The bodyside liner 28 may be of different shapes, including rectangular, hourglass, or any other shape. The bodyside liner 28 may be suitably compliant, soft feeling, and non-irritating to the wearer's skin and may be the same as or less hydrophilic than the absorbent body 34 to permit body exudates to readily penetrate through to the absorbent body 34 and provide a relatively dry surface to the wearer.

The bodyside liner 28 may be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics may be used for the bodyside liner 28. The bodyside liner 28 may include a woven fabric, a nonwoven fabric, a polymer film, a film-fabric laminate or the like, as well as combinations thereof. Examples of a nonwoven fabric may include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, may include more than one layer of fabrics, films, and/or webs, as well as combinations thereof. For example, the bodyside liner 28 may include a support layer and a projection layer that may be hydroentagled. The projection layer may include hollow projections, such as those disclosed in U.S. Pat. No. 9,474,660 to Kirby, Scott S. C. et al.

For example, the bodyside liner 28 may be composed of a meltblown or spunbond web of polyolefin fibers. Alternatively, the bodyside liner 28 may be a bonded-carded web composed of natural and/or synthetic fibers. The bodyside liner 28 may be composed of a substantially hydrophobic material, and the hydrophobic material may, optionally, be treated with a surfactant or otherwise processed to impart a desired level of wettability and hydrophilicity. The surfactant may be applied by any conventional mechanism, such as spraying, printing, brush coating or the like. The surfactant may be applied to the entire bodyside liner 28, or the surfactant may be selectively applied to particular sections of the bodyside liner 28.

In an example embodiment, a bodyside liner 28 may be constructed of a non-woven bicomponent web. The non-woven bicomponent web may be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an example embodiment, a bodyside liner 28 may be a spunbond substrate with a basis weight from about 10 or 12 to about 15 or 20 gsm. In an example embodiment, a bodyside liner 28 may be a 12 gsm spunbond-meltblown-spunbond substrate having 10% meltblown content applied between the two spunbond layers.

Although the outer cover 26 and bodyside liner 28 may include elastomeric materials, it is contemplated that the outer cover 26 and the bodyside liner 28 may be composed of materials which are generally non-elastomeric. In an example embodiment, the bodyside liner 28 may be stretchable, and more suitably elastic. In an example embodiment, the bodyside liner 28 may be suitably stretchable and more suitably elastic in at least the lateral or circumferential direction of the absorbent article 10. In other example aspects, the bodyside liner 28 may be stretchable, and more suitably elastic, in both the lateral and the longitudinal directions 32, 30, respectively.

In an example embodiment, the absorbent article 10 may include a pair of containment flaps 50, 52. The containment flaps 50, 52 may be formed separately from the absorbent chassis 11 and attached to the chassis 11 or may be formed integral to the chassis 11. In some example embodiments, the containment flaps 50, 52 may be secured to the chassis 11 of the absorbent article in a generally parallel, spaced relation with each other laterally inward of the leg openings to 10 provide a barrier against the flow of body exudates. One containment flap 50 may be on a first side of the longitudinal axis 29 and the other containment flap 52 may be on a second side of the longitudinal axis 29. In an example embodiment, the containment flaps 50, 52 may extend generally in a longitudinal direction 30 from the front waist region 12 of the absorbent article 10 through the crotch region 16 to the rear waist region 14 of the absorbent article 10. In some example embodiments, the containment flaps 50, 52 may extend in a direction substantially parallel to the longitudinal axis 29 of the absorbent article 10, however, in other example embodiments, the containment flaps 50, 52 may be curved, as is known in the art.

In example embodiments where the containment flaps 50, 52 are coupled to the chassis 11, the containment flaps 50, 52 may be bonded to the bodyside liner 28 with a barrier adhesive connecting the projections portion 66 to the body facing surface 19 of the chassis 11, or the containment flaps 50, 52 may be bonded to the outer cover 26 with a barrier adhesive in some example embodiments where the bodyside liner 28 does not extend the full lateral width of the outer cover 26. Of course, the containment flaps 50, 52 may be bonded to other components of the chassis 11 and may be bonded with other suitable mechanism other than a barrier adhesive. The containment flaps 50, 52 may be constructed of a fibrous material which may be similar to the material forming the bodyside liner 28. Other conventional materials, such as polymer films, may also be employed.

The containment flaps 50, 52 may each include a base portion 64 and a projection portion 66. The base portion 64 may be bonded to the chassis 11, for example, to the bodyside liner 28 or the outer cover 26 as mentioned above. The base portion 64 may include a proximal end 64a and a distal end 64b. The projection portion 66 may be separated from the base portion 64 at the proximal end 64a of the base portion 64. As used in this context, the projection portion 66 may be separated from the base portion 64 at the proximal end 64a of the base portion 64 in that the proximal end 64a of the base portion 64 defines a transition between the projection portion 66 and the base portion 64. The proximal end 64a of the base portion 64 may be located near the barrier adhesive. In some example embodiments, the distal ends 64b of the base portion 64 may laterally extend to the respective longitudinal side edges 18, 20 of the absorbent article 10. In other example embodiments, the distal ends 64b of the base portion 64 may end laterally inward of the respective longitudinal side edges 18, 20 of the absorbent article 10. The containment flaps 50, 52 may also each include a projection portion 66 that is configured to extend away from the body facing surface 19 of the chassis 11 at least in the crotch region 16 when the absorbent article 10 are in a relaxed configuration. The containment flaps 50, 52 may include a tack-down region 71 in either or both of the front waist region 12 and the rear waist region 14 where the projection portion 66 is coupled to the body facing surface 19 of the chassis 11.

It is contemplated that the containment flaps 50, 52 may be of various configurations and shapes, and may be constructed by various methods. For example, the containment flaps 50, 52 of FIG. 1 depict a longitudinally extending containment flap 50, 52 with a tack-down region 71 in both the front and rear waist regions 12, 14 where the projection portion 66 of each containment flap 50, 52 is tacked down to the bodyside liner 28 towards or away from the longitudinal axis 29 of the absorbent article 10. However, the containment flaps 50, 52 may include a tack-down region 71 where the projection portion 66 of each of the containment flaps 50, 52 is folded back upon itself and coupled to itself and the bodyside liner 28 in a “C-shape” configuration, as is known in the art and described in U.S. Pat. No. 5,895,382 to Robert L. Popp et al. As yet another alternative, it is contemplated that the containment flaps 50, 52 may be constructed in a “T-shape” configuration, such as described in U.S. Pat. No. 9,259,362 to Robert L. Popp et al. Such a configuration may also include a tack-down region 71 in either or both of the front and rear waist regions 12, 14, respectively. Of course, other configurations of containment flaps 50, 52 may be used in the absorbent article 10 and still remain within the scope of this disclosure.

The containment flaps 50, 52 may include one or more flap elastic members 68, such as the two flap elastic strands depicted in FIG. 1. Suitable elastic materials for the flap elastic members 68 may include sheets, strands or ribbons of natural rubber, synthetic rubber, or thermoplastic elastomeric materials. Of course, while two elastic members 68 are shown in each containment flap 50, 52, it is contemplated that the containment flaps 50, 52 may be configured with one or three or more elastic members 68. Alternatively or additionally, the containment flaps 50, 52 may be composed of a material exhibiting elastic properties itself.

The flap elastic members 68, as illustrated in FIG. 1, may have two strands of elastomeric material extending longitudinally in the projection portion 66 of the containment flaps 50, 52, in generally parallel, spaced relation with each other. The elastic members 68 may be within the containment flaps 50, 52 while in an elastically contractible condition such that contraction of the strands gathers and shortens the projection portions 66 of the containment flaps 50, 52 in the longitudinal direction 30. As a result, the elastic members 68 may bias the projection portions 66 of the containment flaps 50, 52 to extend away from the body facing surface 45 of the absorbent assembly 44 in a generally upright orientation of the containment flaps 50, 52, especially in the crotch region 16 of the absorbent article 10, when the absorbent article 10 is in a relaxed configuration.

During manufacture of the containment flaps 50, 52 at least a portion of the elastic members 68 may be bonded to the containment flaps 50, 52 while the elastic members 68 are elongated. The percent elongation of the elastic members 68 may be, for example, about 110% to about 350%. The elastic members 68 may be coated with adhesive while elongated to a specified length prior to attaching to the elastic members 68 to the containment flaps 50, 52. In a stretched condition, the length of the elastic members 68 which have adhesive coupled thereto may provide an active flap elastic region 70 in the containment flaps 50, 52, as labeled in FIG. 1, which will gather upon relaxation of the absorbent article 10. The active flap elastic region 70 of containment flaps 50, 52 may be of a longitudinal length that is less than the length LAA of the absorbent article 10. In this exemplary method of bonding the elastic members 68 to the containment flaps 50, 52, the portion of the elastic members 68 not coated with adhesive will retract after the elastic members 68 and the absorbent article 10 are cut in manufacturing to form an individual absorbent article 10. As noted above, the relaxing of the elastic members 68 in the active flap elastic region 70 when the absorbent article 10 is in a relaxed condition may cause each containment flap 50, 52 to gather and cause the projection portion 66 of each containment flap 50, 52 to extend away from the body facing surface 19 of the chassis 11 (e.g., the body facing surface 45 of the absorbent assembly 44 or the body facing surface 56 of the bodyside liner 28).

Of course, the elastic members 68 may be bonded to the containment flaps 50, 52 in various other ways as known by those of skill in the art to provide an active flap elastic region 70, which is within the scope of this disclosure. Additionally, the active flap elastic regions 70 may be shorter or longer than depicted herein, including extending to the front waist edge 22 and the rear waist edge 24, and still be within the scope of this disclosure.

Leg elastic members 60, 62 may be secured to the outer cover 26, such as by being bonded thereto by laminate adhesive, generally laterally inward of the longitudinal side edges, 18 and 20, of the absorbent article 10. The leg elastic members 60, 62 may form elasticized leg cuffs that further help to contain body exudates. In an example embodiment, the leg elastic members 60, 62 may be disposed between inner and outer layers (not shown) of the outer cover 26 or between other layers of the absorbent article 10, for example, between the base portion 64 of each containment flap 50, 52 and the bodyside liner 28, between the base portion 64 of each containment flap 50, 52 and the outer cover 26, or between the bodyside liner 28 and the outer cover 26. The leg elastic members 60, 62 may be one or more elastic components near each longitudinal side edge 18, 20. For example, the leg elastic members 60, 62 as illustrated herein may each include two elastic strands. A wide variety of elastic materials may be used for the leg elastic members 60, 62. Suitable elastic materials may include sheets, strands or ribbons of natural rubber, synthetic rubber, or thermoplastic elastomeric materials. The elastic materials may be stretched and secured to a substrate, secured to a gathered substrate, or secured to a substrate and then elasticized or shrunk, for example, with the application of heat, such that the elastic retractive forces are imparted to the substrate. Additionally, it is contemplated that the leg elastic members 60, 62 may be formed with the containment flaps 50, 52, and then attached to the chassis 11 in some example embodiments. Of course, the leg elastic members 60, 62 may be omitted from the absorbent article 10 without departing from the scope of this disclosure.

In an example embodiment, the absorbent article 10 may have one or more elasticated waist members 54. The elasticated waist member(s) 54 may be disposed in the rear waist region 14 as illustrated in FIG. 1, or in both the rear waist region 14 and the front waist region 12. Although generally described in the present disclosure with reference to a singular elasticated waist member, it should be understood that such description applies equally to each elasticated waist member in example embodiments which contain multiple elasticated waist members 54. As will be discussed in more detail below, the elasticated waist member 54 may help contain and/or absorb body exudates, especially low viscosity fecal matter, and as such, may be preferred to be in the rear waist region 14. An elasticated waist member 54 in the front waist region 12 may help contain and/or absorb body exudates, such as urine, in the front waist region 12. Although not as prevalent as in the rear waist region 14, in some circumstances, fecal material may also spread to the front waist region 12, and thus, an elasticated waist member 54 disposed in the front waist region 12 may help contain and/or absorb body exudates as well.

The elasticated waist member 54 may be comprised of a variety of materials. In a preferred example embodiment, the elasticated waist member 54 may be comprised of a spunbond-meltblown-spunbond (“SMS”) material. However, it is contemplated that the elasticated waist member 54 may be comprised of other materials, such as a spunbond-film-spunbond (“SFS”), a bonded carded web (“BCW”), or any non-woven material. In some example embodiments, the elasticated waist member 54 may be comprised of a laminate of more than one of these exemplary materials, or other materials. In some example embodiments, the elasticated waist member 54 may be comprised of a liquid impermeable material, for example a film material. In some example embodiments, the elasticated waist member 54 may be comprised of a material coated with a hydrophobic coating. The basis weight of the material forming the elasticated waist member 54 may vary, however, in a preferred example embodiment, the basis weight may be between about 8 gsm to about 120 gsm, not including the elastic members 86 in the elasticated waist member 54. More preferably, the basis weight of the material comprising the elasticated waist member 54 may be between about 10 gsm to about 40 gsm, and even more preferably, between about 15 gsm to about 25 gsm.

The elasticated waist member 54 may include a first longitudinal side edge 72, a second longitudinal side edge 74, a waist member first end edge, and a waist member second end edge joining the first longitudinal edge 72 and the second longitudinal edge 74. The first longitudinal side edge 72 may be opposite from the second longitudinal side edge 74. The distance between the first longitudinal side edge 72 and the second longitudinal side edge 74 may define a width of the elasticated waist member 54 in the lateral direction 32. Although not depicted, in some example embodiments, the first longitudinal side edge 72 may substantially align with the first longitudinal side edge 18 of the absorbent article 10. Similarly, in some example embodiments, the second longitudinal side edge 74 may align with the second longitudinal side edge 20 of the absorbent article 10. As illustrated in FIG. 1, the elasticated waist member 54 may be configured such that the first longitudinal side edge 72 may be disposed laterally outward of the proximal end 64a of the base portion 64 of the containment flap 50. Similarly, the elasticated waist member 54 may be configured such that the second longitudinal side edge 74 may be disposed laterally outward of the proximal end 64a of the base portion 64 of the containment flap 52.

In preferred example embodiments, the elasticated waist member 54 may include at least one elastic member 86. In some example embodiments, the elasticated waist member 54 may include multiple elastic members 86, such as five elastic members 86. Of course, it is contemplated that the elasticated waist member 54 may include other numbers of elastic members 86, such as three, four, six, eight, or ten elastic members, and in some example embodiments, no elastic members 86. The elastic member 86 may span substantially from the first longitudinal side edge 72 to the second longitudinal side edge 74 of the elasticated waist member 54. In some example embodiments, the elastic members 86 may be spaced evenly in the longitudinal direction 30. At least one of the elastic members 86 may be disposed located near the waist member second end edge of the elasticated waist member 54.

A wide variety of elastic materials may be used for the elastic member(s) 86 in the elasticated waist member 54. Suitable elastic materials may include sheets, strands or ribbons of natural rubber, synthetic rubber, thermoplastic elastomeric materials, or elastic foams. The elastic materials may be stretched and secured to a substrate forming the elasticated waist member 54, secured to a gathered substrate, or secured to a substrate and then elasticized or shrunk, for example, with the application of heat, such that the elastic retractive forces are imparted to the substrate forming the elasticated waist member 54.

In some example embodiments, the elasticated waist member 54 may be disposed on the body facing surface 45 of the absorbent assembly 44. In some example embodiments, such as in example embodiments illustrated in FIG. 1, the elasticated waist member 54 may be disposed on the body facing surface 56 of the bodyside liner 28.

In various example embodiments, the elasticated waist member 54 may also include a proximal portion (not shown) and a distal portion (not shown). The proximal portion may be coupled to the body facing surface 19 of chassis 11 (e.g., the body facing surface 45 of the absorbent assembly 44 or the body facing surface 56 of the bodyside liner 28) whereas the distal portion or at least a portion of the distal portion of the elasticated waist member 54 may be free to move with respect to the chassis 11 and the absorbent assembly 44 when the absorbent article 10 is in the relaxed configuration. A first fold (not shown) may separate the proximal portion from the distal portion in the various example embodiments of the elasticated waist member 54 discussed herein. As used in this context, the first fold separates the proximal portion from the distal portion in that the first fold defines a transition between the proximal portion and the distal portion in the elasticated waist member material and the elasticated waist member 54 as a whole. In alternate example embodiments (not shown), the proximal portion and the distal portion may be made from separate materials which are attached to each other such as, for example, in the area of the first fold or in lieu of the first fold. The physical form of the attachment may be, for example, by way of a butt seam or a lap seam.

The proximal portion of such an elasticated waist member 54 may be coupled to the body facing surface 19 of the chassis 11 with an adhesive, and in some example embodiments, the proximal portion may be coupled to the body facing surface 45 of the absorbent assembly 44. In some example embodiments, the proximal portion of the elasticated waist member 54 may be coupled to the body facing surface 56 of the bodyside liner 28. However, in some example embodiments, the proximal portion of the elasticated waist member 54 may be coupled to the body facing surface 58 of the rear waist panel 15. The proximal portion may be coupled to the body facing surface 45 of the absorbent assembly 44 with adhesive along the entire length of the proximal portion in the longitudinal direction 30. However, it can be contemplated that only a portion of the proximal portion in the longitudinal direction 30 may be coupled to the body facing surface 45 of the absorbent assembly 44. Of course, it is contemplated that the proximal portion of the elasticated waist member 54 may be coupled to the body facing surface 19 of the chassis 11 or the body facing surface 45 of the absorbent assembly 44 by means other than an adhesive, such as by pressure bonding, ultrasonic bonding, thermal bonding, and combinations thereof. In preferred example embodiments, the proximal portion is coupled to the body facing surface 19 of the chassis 11 in the lateral direction 32 in a constant fashion along the lateral axis 31, as opposed to an intermittent fashion, such that a barrier to body exudates is formed between the proximal portion and the body facing surface 19 of the chassis 11.

The proximal portion of the elasticated waist member 54 may include a longitudinal length measured in the longitudinal direction 30 along the longitudinal axis 29 that is shorter than a longitudinal length of the distal portion of the elasticated waist member 54 (not shown). However, in some example embodiments, the longitudinal length of the proximal portion may be substantially equal to or larger than the longitudinal length of the distal portion of the elasticated waist member 54. It can be appreciated that the relative longitudinal lengths of the proximal portion and the distal portion may be varied between example embodiments of the elasticated waist member 54 without departing from the scope of this disclosure.

In such example embodiments of an elasticated waist member 54, because the distal portion of the elasticated waist member 54 can freely move with respect to the absorbent assembly 44 when the absorbent article 10 is in the relaxed configuration, the distal portion may assist with providing a containment pocket when the absorbent article 10 is in the relaxed configuration when being worn by the wearer. The containment pocket may assist with providing a barrier to contain and/or absorb body exudates. The first longitudinal side edge 72 may be disposed laterally outward of the proximal end 64a of the base portion 64 of the containment flap 50, and thus, the pocket may extend laterally outward of the proximal end 64a of the containment flap 50. Similarly, the second longitudinal side edge 74 may be disposed laterally outward of the proximal end 64a of the base portion 64 of the containment flap 52 and the pocket may extend laterally outward of the proximal end 64a of the containment flap 52. Such a configuration provides elasticated waist member 54 with a wide containment pocket to contain and/or absorb body exudates. To help prevent lateral flow of body exudates that are contained by the containment pocket of the elasticated waist member 54, the distal portion of the elasticated waist member 54 may be bonded to the proximal portion of the elasticated waist member 54 and/or the body facing surface 19 of the chassis 11 near the first and second longitudinal side edges 72, 74, respectively. For example, FIG. 1 depicts tack-down regions 84 where the distal portion of the elasticated waist member 54 may be bonded to the proximal portion of the elasticated waist member 54 and/or the body facing surface 19 of the chassis 11 near the first and second longitudinal side edges 72, 74, respectively.

As depicted in FIG. 1, in some example embodiments the elasticated waist member 54 may be disposed on the body facing surface 19 of the chassis 11 such that a gap is provided between the second end edge 42 of the absorbent body 34 and the waist member second end edge of the distal portion of the elasticated waist member 54. By providing a gap, the containment may have a greater void volume for body exudates. Additionally, it is believed that gap can help body exudates enter the containment pocket of the elasticated waist member 54.

The elasticated waist member 54 may be disposed to be coupled to the chassis 11 by being placed either over the containment flaps 50, 52 or under the containment flaps 50, 52. More specifically, as shown in FIG. 1, the elasticated waist member 54 may be disposed on the body facing surface 19 of the chassis 11 such that the proximal portion of the elasticated waist member 54 is disposed over the base portion 64 of the first and the second containment flaps 50, 52, respectively. Alternatively, the elasticated waist member 54 may be disposed on the body facing surface 19 of the chassis 11 such that the proximal portion of the elasticated waist member 54 is disposed under the base portion 64 of the first and the second containment flaps 50, 52, respectively. Both configurations can provide advantages to the functioning of the elasticated waist member 54 to contain and/or absorb body exudates.

Example embodiments where the proximal portion of the elasticated waist member 54 is disposed over the base portion 64 of the containment flaps 50, 52 can provide the advantage that the containment flaps 50, 52 assist the distal portion of the elasticated waist member 54 extend away from the body facing surface 45 of the absorbent assembly 44 when the absorbent article 10 is applied to the wearer. This is especially relevant where the proximal portion of the elasticated waist member 54 has a shorter longitudinal length than the distal portion of the elasticated waist member 54. For example, when the proximal portion is shorter than the distal portion, the flap elastics 68 in the projection portion 66 of the containment flaps 50, 52 can provide an opening force on the distal portion of the elasticated waist member 54 when the absorbent article 10 is in the relaxed configuration and applied to the wearer, thus helping the distal portion extend away from the body facing surface 45 of the absorbent assembly 44 and opening the containment pocket. In some example embodiments, the containment pocket may be additionally or alternatively opened by configuring the containment flaps 50, 52 to have an active flap elastic region 70 that longitudinally overlaps with the distal portion of the elasticated waist member 54 when the absorbent article 10 is in the stretched, laid flat configuration, such as illustrated in FIG. 1. Additionally or alternatively, the containment pocket of the elasticated waist member 54 may be opened by configuring the containment flaps 50, 52 to have a tack-down region 71 that does not extend to a distal edge of the distal portion of the elasticated waist member 54, such as illustrated in FIG. 1. However, such a configuration of the tack-down region 71 is not required, and in some example embodiments, the tack-down region 71 may extend from the rear waist edge 24 past the distal edge of the distal portion of the elasticated waist member 54.

Example embodiments where the proximal portion of the elasticated waist member 54 is disposed under the base portion 64 of the containment flaps 50, 52 can provide the advantage of having the containment pocket formed by the elasticated waist member 54 be free from the projection portion 66 of the containment flaps 50, 52. Both the base portion 64 and the projection portion 66 of each containment flap 50, 52 may be coupled to the body facing surface 55 of the elasticated waist member 54. As a result, body exudates may more freely spread through the full width of the containment pocket created by the elasticated waist member 54. Additionally, the coupling of the base portion 64 of the containment flaps 50, 52 to the outer cover 26 (or in some example embodiments to the bodyside liner 28) may create a longitudinal barrier to the flow of body exudates out of the containment pocket for exudates that spread laterally beyond the location of a barrier adhesive connecting the projection portion 66 of the flaps 50, 52 to the body facing surface 19 of the chassis 11. In some example embodiments, the tack-down region 71 of the projection portion 66 of each of the containment flaps 50, 52 may longitudinally overlap with the distal portion of the elasticated waist member 54. In some example embodiments, the tack-down region 71 of projection portion 66 of each of the containment flaps 50, 52 may extend to the distal edge of the elasticated waist member 54 to further assist in containing exudates within the containment pocket created by the elasticated waist member 54. For instance, the containment pocket and other components of absorbent article 10 may be formed in the same or similar manner to that described in U.S. Pat. No. 10,159,610, which is incorporated by reference herein in its entirety for all purposes.

In example embodiments, the absorbent article 10 may include a fastening system. The fastening system may include one or more back fasteners 91 and one or more front fasteners 92. The example embodiments being shown in FIG. 1 depict example embodiments with one front fastener 92. Portions of the fastening system may be included in the front waist region 12, rear waist region 14, or both.

The fastening system may be configured to secure the absorbent article 10 about the waist of the wearer in a fastened condition and help maintain the absorbent article 10 in place during use. In an example embodiment, the back fasteners 91 may include one or more materials bonded together to form a composite ear as is known in the art. For example, the composite fastener may be composed of a stretch component 94, a nonwoven carrier or hook base 96, and a fastening component 98, as labeled in FIG. 1. In some example embodiments, the elasticated waist member 54 may laterally extend to the back fasteners 91, and/or to each of the longitudinal side edges 18, 20 of the absorbent article 10. In some example embodiments, the elasticated waist member 54 may be coupled to the stretch component 94 of the back fasteners 91, either directly or indirectly.

According to some example embodiments of the present disclosure, bodyside liner 28 of the article 10 may further include zones with different morphological features. In various example embodiments, the morphological features may include one or more of: discrete perforated zones; discrete depression and/or protrusion zones, e.g., with depressions and/or hollow protrusions (such as those formed by embossing or other such web-modification processes); discrete apertures (whether integrally formed during web formation or formed through post web-formation processes); and/or discrete filled protrusions extending above, or below depending on orientation, a generally planar surface of the web.

As a particular example, bodyside liner 28 may include a first feature zone 21 with one or more first features 23 and a second feature zone 25 with one or more second features 27. In the example embodiment shown in FIG. 1, the features 23, 27 may be apertures that extend through bodyside liner 28. The apertures 23, 27 may assist with transferring body exudates through the bodyside liner 28 into interior portions of the article 10 where the exudates are stored and disposed away from a wearer's skin. However, while described in greater detail below in the context of apertures, it will be understood that each of first and second feature zones 21, 25 may include one or more alternative structural features in other example embodiments. For instance, each of first and second feature zones 21, 25 may include one or more of embossments, projections, depressions, perforations, protrusions, recesses, apertures, and the like. Thus, each of first and second feature zones 21, 25 may include discrete structural features 23, 27 on bodyside liner 28, and the structural features 23 in first feature zone 21 may be spaced apart from the structural features 27 in second feature zone 25 on bodyside liner 28. In certain example embodiments, the structural features 23 in first feature zone 21 may be sized and/or formed differently than the structural features 27 in second feature zone 25. Thus, e.g., the structural features 23 in first feature zone 21 may provide different performance characteristics than the structural features 27 in second feature zone 25. Moreover, each of first and second feature zones 21, 25 may be separately arranged, sized, shaped, and/or configured to provide a respective performance characteristic therein.

Portions of the bodyside liner 28 including the apertures 27 may be particularly suited to transferring and trapping low-viscosity fecal matter away from a wearer's skin. Such an effect may help to maintain comfort and skin health of a wearer by preventing prolonged contact between the fecal matter and the skin of a wearer of article 10. Portions of the bodyside liner 28 including the apertures 27, or other relatively large apertures, may be less desirable for management of urine exudate. For example, where apertures 27 are large enough or plentiful enough to provide a relatively large open area of the liner 28, such apertures 27 provide an avenue for urine to seep back to the body facing surface 19 of the article 10 and thus in contact with a wearer's skin-potentially causing discomfort and/or skin health issues.

In at least some example embodiments, the second feature zone 25 may be disposed in a localized region of the article 10, as shown in FIG. 1. For example, the second feature zone 25 may have a longitudinal extent that is less than the longitudinal extent LAA of the article 10. More specifically, the second feature zone 25 may have a longitudinal extent that less than seventy percent (70%), such as less than sixty percent (60%), such as less than fifty percent (50%), such as about less than forty-seven percent (47%), of the longitudinal extent LAA of the article 10. Additionally, the second feature zone 25 may have a lateral extent that is less than the lateral extent WAA of the article 10. In some of these example embodiments, the second feature zone 25 may be disposed wholly between proximal ends 64a of base portions 64 of the containment flaps 50, 52. In some example embodiments, the second feature zone 25 may be located wholly within the crotch region 16. In other example embodiments, the second feature zone 25 may be located wholly within the rear waist region 14. In still further example embodiments, the second feature zone 25 may span both a portion of the crotch region 16 and the rear waist region 14. In still further example embodiments, the second feature zone 25 may be located between no less than forty percent (40%) and no greater than eighty percent (80%) of the longitudinal extent LAA of the article 10 from the front waist edge 22. In certain example embodiments, the second feature zone 25 may also extend across the lateral axis 31, e.g., such that the second feature zone 25 at least partially overlaps the lateral axis 31 within the crotch region 16. The crotch region 16 and the rear waist region 14 are the locations within the article 10 where fecal matter is typically received.

Portions of the bodyside liner 28 forming the first feature zone 21 may be particularly suited to handling fluids, such as urine,—for example, transferring and trapping liquid away from the skin of a wearer of the article 10. Such an effect may help to maintain comfort and skin health of the wearer by preventing prolonged contact between the urine and the skin of the wearer of the article 10. As noted above, the first feature zone 21 may accomplish such function through the use of individual structural features or any combination of structural features. In at least some example embodiments, the structural features 23 of the first feature zone 21 include apertures. Where the features 23 include apertures, such apertures may be relatively small apertures for management of urine exudate.

Accordingly, in at least some example embodiments, the first feature zone 21 may be disposed in a localized region of the article 10, as shown in FIG. 1. For example, the first feature zone 21 may have a longitudinal extent that is less than the longitudinal extent LAA of the article 10. More specifically, the first feature zone 21 may have a longitudinal extent that is less half, or less than a third, or less than a quarter, of the longitudinal extent LAA of the article 10. Additionally, the first feature zone 21 may have a lateral extent that is less than the lateral extent WAA of the article 10. In some of these example embodiments, the first feature zone 21 may be disposed wholly between proximal ends 64a of base portions 64 of the containment flaps 50, 52. In some example embodiments, the first feature zone 21 may be located wholly within the crotch region 16. The crotch region 16 is the location within the article 10 where urine is typically received.

Three-Dimensional Web with Nodes:

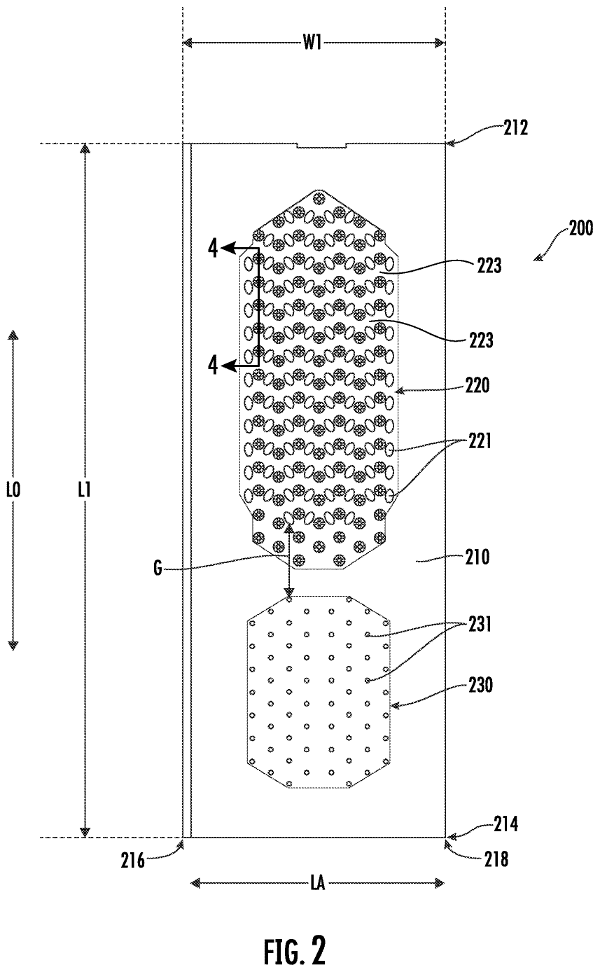

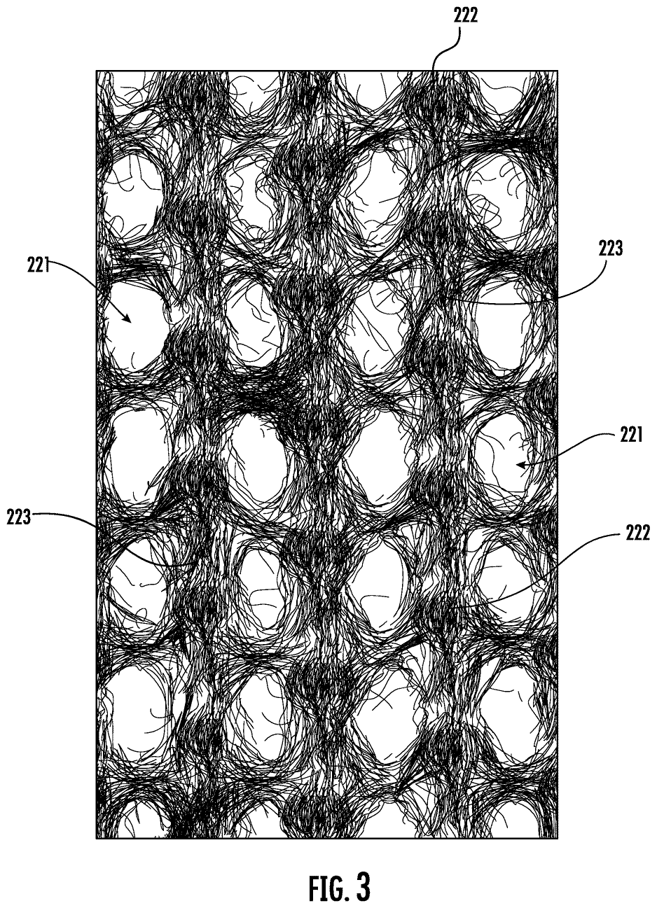

FIG. 2 is a top, plan view of a nonwoven material 200 according to an example embodiment of the present disclosure. In FIG. 2, nonwoven material 200 is shown in a flat or planar configuration. FIG. 3 is an image of a first feature zone 220 of nonwoven material 200. Nonwoven material 200 may be used in absorbent article 10, e.g., as bodyside liner 28, Thus, nonwoven material 200 is described in greater detail below in the context of absorbent article 10. However, it will be understood that nonwoven material 200 may be used in any other article or garment in alternative example embodiments. For instance, nonwoven material 200 may be used in diapers, diaper pants, training pants, youth pants, swim pants, feminine hygiene products, including, but not limited to, menstrual pads or pants, incontinence products, medical garments, surgical pads and bandages, other personal care or health care garments, e.g., as an inner liner facing a wearer of such products, and wipes. As discussed in greater detail below, nonwoven material 200 may advantageously include three-dimensional topography with desirable physical characteristics.

As shown in FIG. 2, nonwoven material 200 includes a nonwoven fibrous web 210, e.g., formed with a plurality of fibers. Fibrous web 210 may define a lateral direction LA and a longitudinal direction LO. The lateral and longitudinal directions LA, LO may be perpendicular to each other. In certain example embodiments, longitudinal direction LO may correspond to the longitudinal direction 30, and lateral direction LA may correspond to the lateral direction 32. Fibrous web 210 may extend between a rear or first end portion 212 and a front or second end portion 214, e.g., along the longitudinal direction LO. Thus, first and second end portions 212, 214 may be spaced apart along the longitudinal direction LO. First end portion 212 of fibrous web 210 may be positioned at or adjacent rear waist region 14 of absorbent article 10, and second end portion 214 of fibrous web 210 may be positioned at or adjacent front waist region 12 of absorbent article 10. Fibrous web 210 may also extend between a first side portion 216 and a second side portion 218, e.g., along the lateral direction LA. Thus, first and second side portions 216, 218 may be spaced apart along the lateral direction LA.

A length L1 of fibrous web 210 may be defined between first and second end portions 212, 214 of fibrous web 210, and a width W1 of fibrous web 210 may be defined between first and second side portions 216, 218 of fibrous web 210. In certain example embodiments, the length L1 of fibrous web 210 may be greater than the width W1 of fibrous web 210. For example, the length L1 of fibrous web 210 may be no less than two times (2×) greater than the width W1 of fibrous web 210, such as no less than three times (3×) greater than the width W1 of fibrous web 210. Thus, fibrous web 210 may be elongated along longitudinal direction LO between the first and second end portions 212, 214.

Nonwoven fibrous web 210 may include at least one feature zone. For instance, as shown in FIG. 3, nonwoven fibrous web 210 may include a first feature zone 220 and a second feature zone 230. First feature zone 220 may be spaced apart from second feature zone 230, e.g., along the longitudinal direction LO. For example, first and second feature zones 220, 230 may be spaced apart by a gap G along the longitudinal direction LO. In certain example embodiments, the gap G may be no less than six millimeters (6 mm) and no greater than one hundred and thirty millimeters (130 mm), such as no less than twelve millimeters (12 mm) and no greater than one hundred millimeters (100 mm), such as no less than twenty-five millimeters (25 mm) and no greater than seventy-five millimeters (75 mm). As shown in FIG. 2, nonwoven fibrous web 210 may be unperforated between first and second feature zones 220, 230, e.g., along the longitudinal direction LO. Thus, e.g., the nonwoven fibrous web 210 may not be processed to include apertures between first and second feature zones 220, 230 along the longitudinal direction LO. In certain example embodiments, a centroid of first feature zone 220 may also be arranged colinear with a centroid of second feature zone 230, e.g., on longitudinal axis 29.

First feature zone 220 may include a plurality of apertures 221 and a plurality of nodes 222, and second feature zone 230 may also include a plurality of apertures 231. Apertures 221 of first feature zone 220 may be larger than apertures 231 of second feature zone 230. For example, an area of each of apertures 221 of first feature zone 220 may be no less than about five millimeters squared (5 mm²) and no greater than about twenty-eight millimeters squared (28 mm²), such as no less than about eight millimeters squared (8 mm²) and no greater than about twenty millimeters squared (20 mm²), such as no less than about nine millimeters squared (9 mm²) and no greater than about twelve millimeters squared (12 mm²). In contrast, an area of each of apertures 231 of second feature zone 230 may be no less than about a quarter millimeter squared (0.25 mm²) and no greater than about five millimeters squared (5 mm²), such as no less than about one millimeter squared (1 mm²) and no greater than about four millimeters squared (4 mm²), such as about two and a half millimeters squared (2.5 mm²). Such sizing differential between apertures 221 of first feature zone 220 and apertures 231 of second feature zone 230 may advantageously facilitate movement of body exudates through nonwoven fibrous web 210. Moreover, the larger apertures 221 of first feature zone 220 may be sized for transferring and trapping low-viscosity fecal matter. Conversely, the smaller apertures 231 of second feature zone 230 may be sized for transferring and trapping urine exudate. In certain example embodiments, apertures 221 may be uniformly distributed throughout first feature zone 220, and apertures 231 may be uniformly distributed throughout second feature zone 230. The area of the apertures 221, 231 within the first and second features zones 220, 230 may be measured using the analysis techniques in the Material Sample Analysis Test Method as described in the Test Methods section herein.

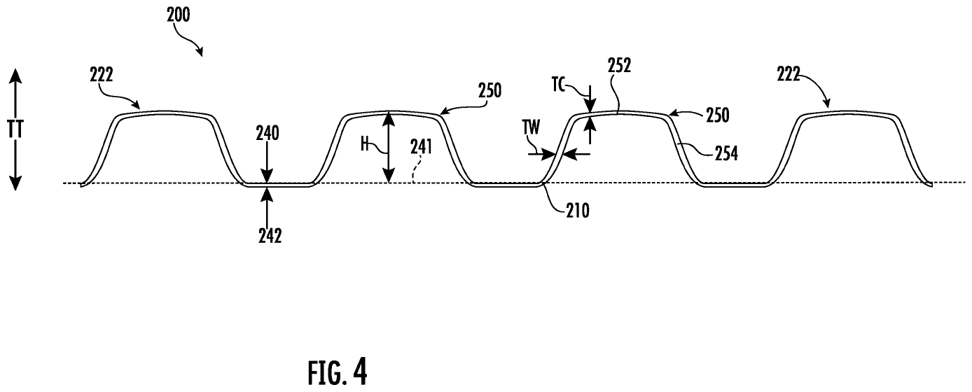

As noted above, nonwoven material 200 may include nodes 222 at first feature zone 220. It will be understood that nonwoven material 200 may include nodes at other locations on nonwoven material 200 in alternative example embodiments. Moreover, it will be understood that, in certain example embodiments, first feature zone 220 may include nodes 222 without apertures 221. As shown in FIG. 3, nonwoven fibrous web 210 may also include a plurality of connecting ligaments 223 at first feature zone 220. As shown in FIG. 4, the nodes 222 may extend away from a base plane 241 on a first surface 240 of the nonwoven material 200. The base plane 241 may be defined as the generally planar region of the first surface 240 of the nonwoven material 200 other than the portion of the nonwoven material 200 forming the nodes 222. In other words, for the example embodiment depicted in FIGS. 2 through 4, the base plane 241 may be formed by the first surface 240 of the nonwoven material 200 that provides the connecting ligaments 223. The nonwoven material 200 may also include a second surface 242. The first surface 240 may be opposite from the second surface 242 on nonwoven fibrous web 210, as depicted in FIG. 4.

The nodes 222 may be configured in a variety of shapes and sizes as will be discussed in further detail below in the discussion of the manufacturing of the nonwoven material 200. In some example embodiments, the nodes 222 may be generally cylindrical in shape. In certain example embodiments, the nodes 222 may be configured to not include any openings or apertures. In certain example embodiments, the nodes 222 may have a height H (as measured in a direction perpendicular to the base plane 241, e.g., from base plane 241 to a distal end portion 250 of node 222) of between about a half millimeter (0.5 mm) to about ten millimeters (10 mm), such as from about one millimeter (1 mm) to about three millimeters (3 mm), such as from about one and one-tenth millimeter (1.1 mm) to about one and five-tenths millimeters (1.5 mm), such as about one and three-tenths millimeters (1.3 mm). Moreover, an average height H of nodes 222 may be no less than twelve hundred microns (1200 μm) and no greater than fourteen hundred microns (1400 μm). It will be understood that the height H of nodes 222 may be measured after the nonwoven material 200 has been wound into manufacturing spool-either after directly being unwound from such a spool or as part of a commercial absorbent article product. The height H of the nodes 222 may be measured using the analysis techniques described in the Node Analysis Test Method described in the Test Methods section herein. In certain example embodiments, a majority of the nodes 222 may each have an area (as measured by the area of the node 222 at the base plane 241) from about five millimeters squared (5 mm²) to about thirty-five millimeters squared (35 mm²), such as from about ten millimeters squared (10 mm²) to about twenty millimeters squared (20 mm²). The nodes 222 may be configured in the first feature zone 220 such that the nodes 222 provide a node density of about one node per square centimeter (1.0 nodes/cm²) to about three nodes per square centimeter (3.0 nodes/cm²). The node area and node density within first feature zone 220 may be measured using the analysis techniques described in the Material Sample Analysis Test Method described in the Test Methods section herein.

As depicted in FIG. 2 and in more detail in FIG. 3, the connecting ligaments 223 may interconnect the nodes 222. Moreover, each connecting ligament 223 may be referred to as extending between only two (2) adjacent nodes 222. In other words, an individual, discrete connecting ligament 223 may not interconnect three (3) or more nodes 222. In this manner, such node and ligament structure differs from an ‘islands-in-the-sea” configuration where the nodes are dispersed between continuously extending and interconnected ‘land areas’. In example embodiments, a majority of nodes 222 may include at least three (3) connecting ligaments 223 connecting to adjacent nodes 222. In certain example embodiments, a majority of nodes 222 may include ten (10) or less connecting ligaments 223 connecting to adjacent nodes 222. In some example embodiments, the nonwoven material 200 may be configured such that a majority of nodes 222 may include three (3) to eight (8) connecting ligaments 223 connecting to adjacent nodes 222. For example, in the example embodiment in FIG. 3, a majority of the nodes 222 include six connecting ligaments 223 that connect to adjacent nodes 222.

The apertures 221 may be areas of the nonwoven fibrous web 210 that have a lower density of fibers of the nonwoven fibrous web 210 in comparison to nodes 222 and connecting ligaments 223. In some example embodiments, the apertures 221 may be substantially devoid of fibers. As used herein, the apertures 221 are to be distinguished from the normal interstitial fiber-to-fiber spacing commonly found in fibrous nonwoven materials. For example, the image in FIG. 2 shows that apertures 221 include a lower density of fibers than adjacent nodes 222 and connecting ligaments 223. The apertures 221 may be formed between the connecting ligaments 223 and the nodes 222. Individual apertures 221 may be disposed between adjacent nodes 222. In certain example embodiments, each aperture 221 may be defined between at least three (3) connecting ligaments 223 and at least three (3) nodes 222. In some example embodiments, individual, discrete apertures 221 may be defined between at least four (4) connecting ligaments 223 and at least four (4) nodes 222. It will be understood that at certain portions of the nonwoven fibrous web 210, such as at edges of at first feature zone 220, the apertures 221 may be defined between less than three (3) connecting ligaments 223 and/or less than three (3) nodes 222.

With reference to FIG. 4, each of nodes 222 may include a cap 252 and a wall 254. Cap 252 may be positioned at distal end portion 250 of each node 222. Wall 254 may extend between first surface 240 and cap 252, e.g., along the transverse direction TT. In certain example embodiments, wall 254 may extend between first surface 240 and distal end portion 250 of each node 222 by the height H of the node 222. Wall 254 and cap 252 may be formed using the processes and apparatuses described below. Utilizing such processes and apparatuses, the nonwoven material 200 with nodes 222 may have advantageous properties relative to known nonwoven materials. As shown in FIG. 4, cap 252 may have a thickness TC. Moreover, the thickness TC of cap 252 may be defined by the fibers of the nonwoven fibrous web 210 forming cap 252, e.g., along the transverse direction TT. Cap 252 may be generally circular in certain example embodiments. In alternative example embodiments, cap 252 may be generally spherical, semi-spherical, triangular, rectangular, etc. Wall 254 may also have a thickness TW. Moreover, the thickness TW of wall 254 may be defined by the fibers of the nonwoven fibrous web 210 forming wall 254, e.g., perpendicular to the transverse direction TT. Wall 254 may be generally cylindrical in certain example embodiments. In alternative example embodiments, wall 254 may be generally triangular prism shaped, rectangular prism shaped, etc.

The thickness TC of caps 252, thickness TW of walls 254, and/or height H of the node 222 may provide advantageous properties for the nonwoven material 200. For instance, a ratio between the average thickness TW of walls 254 of nodes 222 to the average thickness TC of caps 232 of nodes 222 may be between 1.1:1 and 1.5:1, such as about 1.2:1. The ratio between the average thickness TW of walls 254 of nodes 222 to the average thickness TC of caps 232 of nodes 222 may correspond to a uniformity ratio for nodes 222. Thus, e.g., the closer that the ratio between the average thickness TW of walls 254 of nodes 222 to the average thickness TC of caps 232 of nodes 222 is to 1:1, the more uniform the thickness of the fibers of the nonwoven fibrous web 210 forming node 222 may be throughout node 222. In example aspects of the present disclosure, the average thickness TW of walls 254 of nodes 222 may be greater than the average thickness TC of caps 232 of nodes 222. The increased average thickness TW of walls 254 relative to the decreased average thickness TC of caps 232 may advantageously increase the strength of the walls 254 of nodes 222 and thus resist compression, e.g., as evidenced by the improved compression linearity described below. Without wishing to be bound to any particular theory, it is believed that the reflected energy of the fluid impacting against the bottom of the blind hole in the forming surface used to form nodes 222 may push fibers of the nonwoven material 200 back towards walls 254 rather than into cap 232 as is the case with open holes in known forming surfaces. Thus, more fibers may be present within the walls 254 of nodes 222 (relative to nodes with thicker caps), thereby increasing the strength of the walls 254. However, it will be understood that moving too many fibers away from caps 232 of nodes 222 may be disadvantageous. Utilizing blind holes to form nodes 222 may advantageously provide the ratio between the average thickness TW of walls 254 of nodes 222 to the average thickness TC of caps 232 of nodes 222 between 1.1:1 and 1.5:1, such as about 1.2:1, thus providing strong, compression resistant nodes 222. A less uniform node 222 may thus have relatively non-uniform thickness throughout node 222. Such non-uniformity may advantageously improve process capability and/or compression resistance.

In another example aspect, it is believed that other comparative dimensions of the nodes 222 contribute to their compression resistance. For example, a ratio of an average height H of nodes 222 to the average thickness TW of walls 254 of nodes 222 may be preferred to be between 3.2:1 and 2.6:1, such as between 3.1:1 and 2.7:1, such as between 3:1 and 2.8:1, such as about 2.9. The ratio between the average height H of nodes 222 to the average thickness TW of walls 254 of nodes 222 may correspond to a slenderness ratio for nodes 222. Thus, e.g., smaller ratios between the average height H of nodes 222 to the average thickness TW of walls 254 of nodes 222 may correspond to stouter nodes 222, e.g., due to less bulk loss at cap 252 resulting in thinner caps 232 and thicker walls 254. Such stoutness may advantageously improve process capability and/or compression resistance.

A combination of the above-described ratios of wall-to-cap thicknesses with the above-described slenderness ratios may represent preferred example embodiments of the material 200. However, it should additionally be understood that such features in isolation are believed to contribute to improved process capability and/or compression resistance. Accordingly, exemplary examples of material 200 include those example embodiments having the above-described wall-to-cap thickness ratios while having slenderness ratios outside of the above-described preferred example ranges. Similarly, exemplary examples of material 200 include those example embodiments having the above-described preferred slenderness ratios while having wall-to-cap thickness ratios outside of the above-described preferred ranges.

In certain example embodiments, the average thickness TW of walls 254 of nodes 222 may be no less than three hundred microns (300 μm) and no greater than six hundred microns (600 μm), such as no less than four hundred and twenty-five microns (425 μm) and no greater than four hundred and seventy-five microns (475 μm), such as about four hundred and fifty microns (450 μm). In certain example embodiments, the average thickness TC of caps 232 of nodes 222 may be no less than two hundred and forty microns (240 μm) and no greater than five hundred microns (500 μm), such as no less than three hundred and forty microns (340 μm) and no greater than four hundred microns (400 μm), such as about three hundred and seventy microns (370 μm). It will be understood that the example dimensions for the thickness TC of caps 252 and the thickness TW of walls 254 are provided by way of example and vary from those listed above in alterative example embodiments. The thickness TC of caps 252 and the thickness TW of walls 254 may be determined using image analysis measurement methods, such as those described in the Node Analysis Test Method.

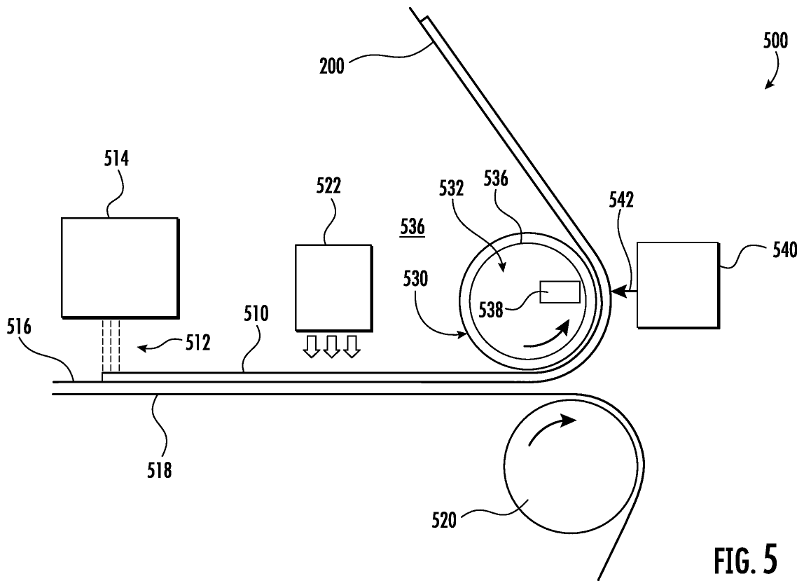

Example processes and apparatuses for forming nonwoven material will now be described. FIG. 5 illustrates an example process and apparatus 500 for how the nonwoven material 200 of the present disclosure may be manufactured. In FIG. 5, a precursor web 510 is provided that includes a plurality of fibers. The precursor web 510 may be formed from a variety of techniques of web forming, such as, but not limited to a wet-laying, a foam-laying, or a carding process. In an example embodiment as depicted in FIG. 5, the precursor web 510 may be formed by a wet-laying process through a fiber and water slurry 512 being deposited from a drum 514 on a precursor forming surface 516. The precursor forming surface 516 as shown in FIG. 5 may be a precursor material, such as a spunbond web- or another pre-formed nonwoven web formed through other web forming technologies, such as meltblowing, thermobonding, wet-laying, air-laying, or hydroentangling. However, it is contemplated that the fiber and water slurry 512 may be deposited directly on a belt, screen, or other surface 518 that provides a precursor forming surface 516.

Where the precursor forming surface 516 is a precursor material, example suitable materials include spunbond materials, bonded-carded materials (for example, such as through air bonded-carded webs (TABCW)), spunlace materials, and any laminates of such materials-including spunbonded-meltblown-spunbond (SMS) webs and similar classes of materials. Preferable basis weights for such precursor materials include materials having basis weights of between about 10 gsm and about 80 gsm, such as between about 10 gsm and about 50 gsm, such as between about 10 gsm and about 30 gsm. Although, other basis weights are contemplated, depending on an end use of the final material. Fibers of the precursor web material may be any suitable fiber type, including being formed of polyolefin materials as is typical for such nonwoven webs in the art. Further, the fibers may have deniers of between about 0.5 and 5.0, such as between about 0.7 and 4.0, such as between about 0.9 and 3.0, such as between about 1.0 and about 2.0. The fibers may have typical staple fiber lengths of less than one hundred millimeters (100 mm). In some example embodiments, the fibers of the precursor web may be similar to those described with respect to the support web 14 in U.S. Pat. No. 9,327,473, titled “Fluid-entangled laminate webs having hollow projections and a process and apparatus for making the same”, to Scott Kirby et al., the entirety of which is hereby incorporated by reference.

As mentioned, the precursor web 510 may be formed according to a variety of techniques (e.g. wet-laying, a foam-laying, or a carding process). The precursor web 510 may preferably have a basis weight of between about 10 gsm and about 80 gsm, such as between about 20 gsm and about 70 gsm, such as between about 30 gsm and about 60 gsm. The precursor web 510 may be formed of typical polyolefin fibers or any suitable polyester-class fibers. Such fibers may preferably have deniers of between about 0.5 and 5.0, such as between about 0.7 and 4.0, such as between about 0.9 and 3.0, such as between about 1.0 and about 2.0. The fibers may have typical staple fiber lengths of less than one hundred millimeters (100 mm). In some example embodiments, the fibers of the precursor web 510 may be similar to those described with respect to the projection web 16 in U.S. Pat. No. 9,327,473 to Scott Kirby et al.

The precursor web may be transferred by a surface 518 driven by a drive roll 520, or other transfer devices known by one of ordinary skill in the art. If the precursor web 510 is formed through a wet-laying process, the precursor web 510 may be dried through known techniques with a dryer 522.

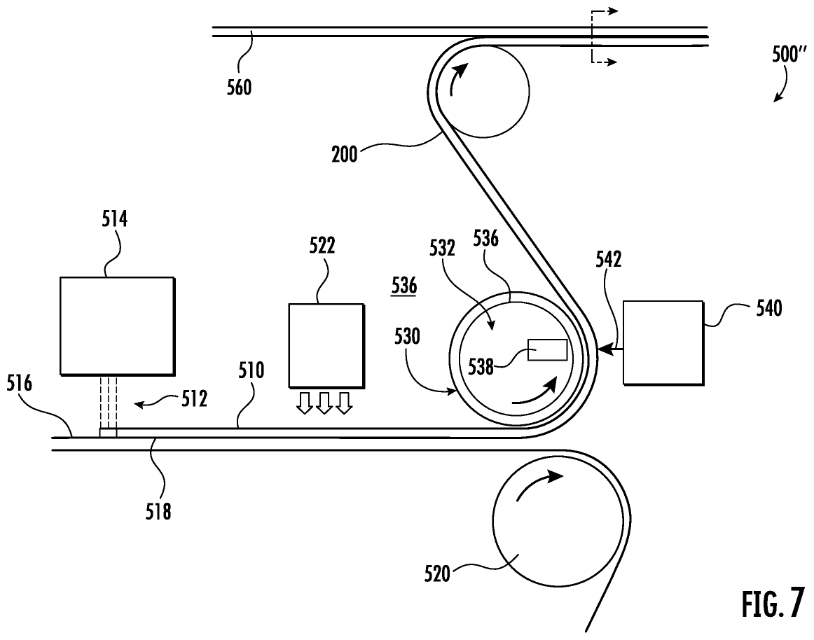

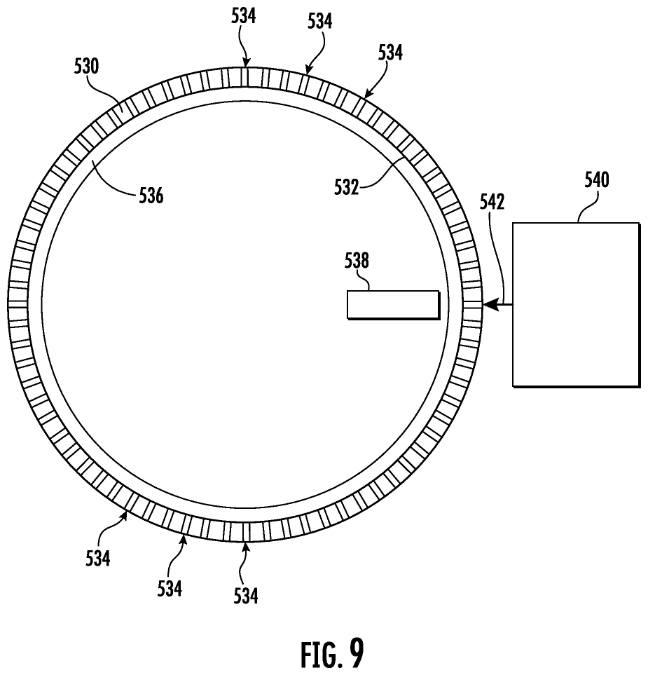

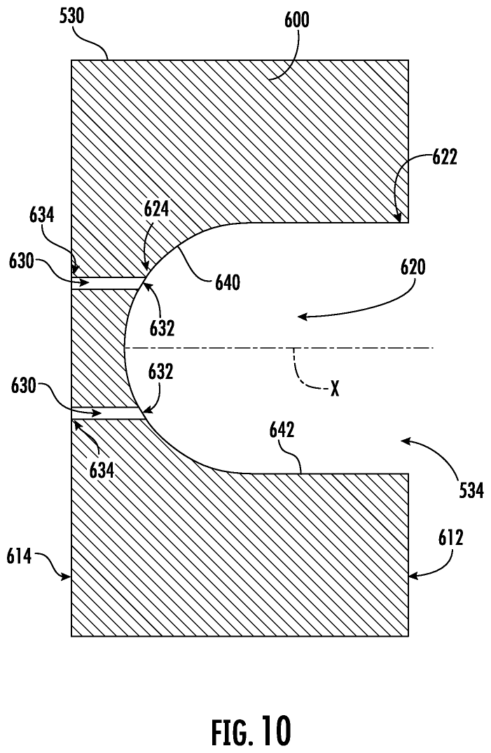

Whether completed off-line or in-line, the precursor web 510 may be transferred to a forming surface 530. The forming surface 530 may be a surface of a texturizing drum 532, such as a forming screen. The forming surface 530 is shown in greater detail in FIGS. 9 and 10. The texturizing drum 532 may rotate as shown in FIG. 7 and may be driven by any suitable drive mechanism (not shown), such as electric motors and gearing, as are well known to those of ordinary skill in the art. The material forming the texturizing drum 532 may be any number of suitable materials commonly used for such forming drums including, but not limited to, sheet metal, plastics and other polymer materials, rubber, etc.

As shown in FIG. 9, the forming surface 530 may include a plurality of forming holes 534. The geometry, spacing, and orientation of the forming holes 534 may correspond to the formation of the nodes 222 in the nonwoven material 200. Moreover, the forming holes 534 may correspond to the shape and pattern of the desired nodes 222 of the nonwoven material 200. Thus, e.g., the forming holes 534 may be arranged in a plurality of columns and/or rows on the forming surface 530. While the forming holes 534 depicted in FIG. 9 are round, it should be understood that any number of shapes and combination of shapes can be used depending on the end use application. Examples of additional or alternative possible forming hole 534 shapes include, but are not limited to, ovals, crosses, squares, rectangles, diamond shapes, hexagons, and other polygons. Further, the forming holes 534 of FIG. 9 are depicted as extending through the forming surface 530 for illustrative purposes only. As will be understood to a greater degree with respect to FIGS. 10 through 15, the forming holes 534 may include blind holes which do not extend completely through the surface of the forming surface 530.

Referring back to FIG. 7, the perforated forming surface 530 may be removably fitted onto and over an optional porous inner drum shell 536 so that different forming surfaces 530 may be used for different end product designs. It will be understood that planar or other shaped forming surface 530 may be utilized in other example embodiments. In the example embodiment of FIGS. 7 and 9, the porous inner drum shell 536 interfaces with a fluid removal system 538 which facilitates pulling the entangling fluid and fibers down into the forming holes 534 in the forming surface 530, thereby forming the nodes 222 in the nonwoven material 200. The porous inner drum shell 536 may also act as a barrier to retard further fiber movement down into the fluid removal system 60 and other portions of the equipment thereby reducing fouling of the equipment. The porous inner drum shell 536 may rotate in the same direction and at the same speed as the texturizing drum 532. In addition, to further control the height of the nodes 222 on the nonwoven material 200, the distance between the inner drum shell 536 and the outer surface of the forming surface 530 can be varied. Generally, the spacing between the inner surface of the forming surface 530 and the outer surface of the inner drum shell 536 may range between about zero millimeters (0 mm) and about five millimeters (5 mm). Other ranges can be used depending on the particular end-use application and the desired features of the nonwoven material 200.

With reference to FIGS. 9 and 10, a depth of the forming holes 534 in the texturizing drum 532 or other projection forming surface 530 may be between one millimeter (1 mm) and ten millimeters (10), such as between about two millimeters (2 mm) and six millimeters (6 mm), such as about four millimeters (4 mm), to produce nodes 222 with the shape most useful in the expected common applications. The forming hole 534 cross-section diameter (or major dimension) may be between about two millimeters (2 mm) and ten millimeters (10 mm), such as between three millimeters (3 mm) and six millimeters (6 mm), such as about five millimeters (5 mm), as measured along the major axis, and a spacing of the forming holes 534 on a center-to-center basis may be between three millimeters (3 mm) and ten millimeters (10 mm), such as between four millimeters (4 mm) and seven millimeters (7 mm). The pattern of the spacing between forming holes 534 may be varied and selected depending upon the particular end use. Some examples of patterns include, but are not limited to, aligned patterns of rows and/or columns, skewed patterns, hexagonal patterns, wavy patterns, and patterns depicting pictures, figures and objects.

The cross-sectional dimensions of the forming holes 534 and the depth of the forming holes 534 may influence the cross-section and height of the nodes 222 produced in the nonwoven material 200. Generally, forming hole 534 shapes with sharp or narrow corners at the leading edge of the forming holes 534 as viewed in the machine direction should be avoided as the sharp or narrow corners may sometimes impair the ability to cleanly remove the nonwoven material 200 from the forming surface 530 without damage to the nodes 222. In addition, the thickness/hole depth in the forming surface 530 will generally tend to correspond to the depth or height of the nodes 222 in the nonwoven material 200. It should be noted, however, that each of the hole depth, spacing, size, shape and other parameters may be varied independently of one another and may be varied based upon the particular end use of the nonwoven material 200 being formed.

Not to be bound by theory, but it is believed that specific aspect ratios of the depth of the forming holes 534 to the diameter (or major dimension) of the forming holes 534 contribute to increased anisotropy of the nodes 222 in the nonwoven material 200. The term “major dimension” is used in the context if the forming holes 534 are not circular in shape, for example, if the forming holes 534 are shaped as an ellipse, the major dimension would be the length of the ellipse along the major axis of the ellipse. It is believed that an aspect ratio of the depth of a forming hole 534 to the diameter (or major dimension) of the forming hole 534 greater than one (1.0) is believed to lead to increased anisotropy of the nodes 222 of the nonwoven material 200. In some example embodiments, the aspect ratio of the depth of the forming holes 534 to the diameter (or major dimension) of the forming holes 534 may be between from one (1.0) to one and two-tenths (1.2). As noted above, increased anisotropy in the nodes 222 in the nonwoven material 200 may provide improved compression properties of the nonwoven material 200.

In the example embodiment of FIGS. 9 and 10, the forming surface 530 is shown in the form of a forming screen placed on texturizing drum 532. It should be appreciated however that other mechanisms may be used to create the forming surface 530. For example, a foraminous belt or wire (not shown) may be used which includes forming holes 534 formed in the belt or wire at appropriate locations. Alternatively, flexible rubberized belts (not shown) which are impervious to the pressurized fluid entangling streams except in the location of the forming holes 534 may be used. Such belts and wires are well known to those of ordinary skill in the art as are the mechanisms for driving and controlling the speed of such belts and wires. A texturizing drum 532 may be more advantageous for formation of the nonwoven material 200 according to the present disclosure because the texturizing drum 532 can be made with the outer surface that is smooth and impervious to entangling fluid at certain locations and does not leave a wire weave pattern on the nonwoven material 200 as wire belts tend to do. In example embodiments where the forming surface 530 forms a portion of a texturizing drum 532 as a forming screen, the forming surface 530 may be achieved through using a variety of techniques. For example, the forming surface 530 and forming holes 534 may be formed by casting, molding, punching, stamping, machining, laser-cutting, water jet cutting, and 3D printing, or any other suitable methodology.

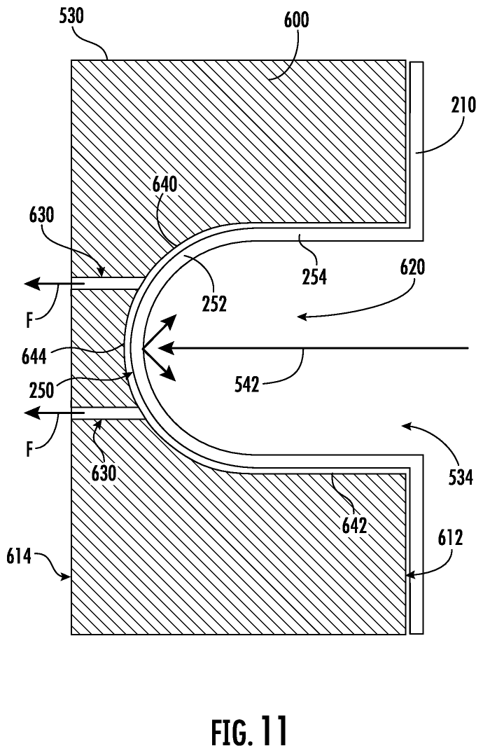

Turning now to FIGS. 10 and 11, the forming surface 530 and forming holes 534 may include features for forming nodes 222 with the advantageous properties described herein. Moreover, forming surface 530 may include a wall 600 with a first surface 612 and a second surface 614. First surface 612 may be positioned opposite second surface 614 on the wall 600. For example, first surface 612 may face away from drum 532 (FIG. 9), and second surface 614 may face towards drum 532. Wall 600 may define formation holes 534. Moreover, as shown, formation holes 534 may include a blind hole 620 and at least one outlet passage 630. Thus, formation holes 534 may be a socket such that formation holes 534 are open at one end of formation holes 534 and at least partially closed (e.g., relative to the open end of formation holes 534) at the opposite end of formation holes 534.

Only one of formation holes 534 is shown in FIGS. 10 and 11; however, it will be understood that other formation holes 534 on forming surface 530 may be formed in the same or similar manner to one of formation holes 534 is shown in FIGS. 10 and 11. As shown, blind hole 620 may extend from a first end portion 622 to a second end portion 624. The first end portion 622 of blind hole 620 may be positioned at first surface 612 of wall 600. Thus, formation hole 534 may be open at first end portion 622 of blind hole 620. Blind hole 620 may also extend into wall 600 towards second surface 614 of wall 600, e.g., such that the second end portion 624 of blind hole 620 is recessed within wall 600. Moreover, e.g., blind hole 620 may extend only partially into wall 600 such that second end portion 624 of blind hole 620 is at least partially closed relative to the open first end portion 622 of blind hole 620 at first surface 612 of wall 600.

Outlet passages 630 may also extend from a first end portion 632 to a second end portion 634. First end portion 632 of outlet passages 630 may be positioned at second end portion 624 of blind hole 620. Thus, e.g., first end portion 632 of outlet passages 630 may be contiguous with blind hole 620 such that fluid within blind hole 620 is flowable out of blind hole 620 through outlet passages 630. Second end portion 634 of outlet passages 630 may be positioned at the second surface 614 of wall 600. Thus, e.g., the fluid from blind hole 620 may exit outlet passages 630 at second end portion 634 of outlet passages 630, e.g., such that the fluid passes through wall 600 from the first surface 612 to the second surface 614 via blind hole 620 and outlet passages 630. In certain example embodiments. The wall 600 may include two (2), three (3), four (4), five (5), six (6), or more outlet passages 630. Outlet passages 630 may be distributed in a circular pattern in certain example embodiments. Outlet passages 630 may also be referred to as “dewatering holes” herein.

As shown in FIG. 11, during formation of nodes 222, nonwoven fibrous web 210 may be positioned on wall 600 at first surface 612 of wall 600. Pressurized fluid stream 542 may impact nonwoven fibrous web 210 in order to direct the nonwoven fibrous web 210 into blind hole 620. The shape and sizing of blind hole 620 may assist with formation of node 222 with advantageous properties. For instance, the pressurized fluid stream 542 may impact nonwoven fibrous web 210 and the material of wall 600 at second end portion 624 of blind hole 620. Moreover, wall 600 may define a forming surface 640 at second end portion 624 of blind hole 620. The nonwoven fibrous web 210 may impact against forming surface 640 at second end portion 624 of blind hole 620 due to the pressurized fluid stream 542, and forming surface 640 may support the nonwoven fibrous web 210, e.g., to prevent bulk loss at distal end portion 250 of node 222. Conversely, in known forming surfaces, the formation holes may be cylindrical along the length of the formation holes such that the distal end portion of the node does not contact any surface and is not supported during formation. The lack of support can cause fibers at the distal end portion of the node to be less formed and concentrated with fibers—with some fibers even escaping the formation hole 534. Furthermore, the pressurized fluid stream may pass directly through the cylindrical formation holes thus dissipating the hydroentangling energy supplied by the pressurized fluid stream resulting in poor formation of the nodes. The formation holes 534 according to example aspects of the present disclosure can advantageously address such drawbacks and form nodes 222 with advantageous properties.

The fluid from pressurized fluid stream 542 may exit formation holes 534 via outlet passages 630. The outlet passages 630 may be arranged to facilitate formation of the nodes 222. For instance, the first and second end portions 622, 624 of blind hole 620 may be spaced apart along a central axis X of blind hole 620. In example embodiments, the central axis X of blind hole 620 may extend perpendicular to a rotational axis of the texturizing drum 532, e.g., such that the central axis X of blind hole 620 extends radially on the texturizing drum 532. The first end portion 632 of each outlet passage 630 may be offset from the central axis X of blind hole 620. Thus, e.g., central axis X may intersect wall 600 at second end portion 624 of blind hole 620 rather than first end portion 632 of outlet passages 630. Such arrangement of outlet passages 630 may assist with supporting the nonwoven fibrous web 210 during formation of node 222, e.g., to prevent bulk loss at distal end portion 250 of node 222. In certain example embodiments, each second end portion 634 of outlet passages 630 may also be offset from the central axis X of blind hole 620. In some of these example embodiments, the outlet passages 630 may extend about parallel to the central axis of socket 620. However, other arrangements of outlet passage 630 are also within the scope of the present subject matter.

Outlet passages 630 and blind hole 620 may be sized differently in order to assist with supporting the nonwoven fibrous web 210 during formation of node 222. For example, a cross-sectional area of blind hole 620 at first end portion 622 of blind hole 620 (e.g., in a plane perpendicular to the central axis X) may be greater than a collective cross-sectional area of outlet passages 630 at first end portion 632 of outlet passage 630 (e.g., in a plane perpendicular to the central axis X). The collective cross-sectional area of outlet passages 630 at first end portion 632 of outlet passage 630 may correspond to the sum of each cross-sectional area of outlet passages 630 at first end portion 632 of outlet passage 630 (e.g., in a plane perpendicular to the central axis X). Moreover, in certain example embodiments, a ratio of the cross-sectional area of blind hole 620 at first end portion 622 of blind hole 620 to the collective cross-sectional area of outlet passages 630 at first end portion 632 of outlet passage 630 may be between 2.75:1 and 11:1, such as between 4:1 and 9:1, such as between 5:1 and 6:1, such as about 5.5:1. In certain example embodiments, the cross-sectional area of blind hole 620 at first end portion 622 of blind hole 620 may be no less than two percent (2%) and no greater than fifteen percent (15%), such as no less than three percent (3%) and no greater than ten percent (10%), such as no less than three and a half percent (3.5%) and no greater than seven percent (7%), of the collective cross-sectional area of outlet passages 630 at first end portion 632 of outlet passage 630. Such differences in cross-sectional area may configure blind hole 620 to support nonwoven fibrous web 210 during formation of node 222 while also configuring outlet passages 630 to drain pressurized fluid stream 542 during formation of node 222. Moreover, the relative size difference between cross-sectional area of blind hole 620 at first end portion 622 of blind hole 620 to the cross-sectional area of outlet passages 630 at first end portion 632 of outlet passage 630 may allow nonwoven fibrous web 210 to deform into blind hole 620 due to the pressurized fluid stream 542; however, the nonwoven fibrous web 210 may be blocked or limited from entering the smaller outlet passages 630.

Forming surface 640 may be shaped to assist with forming node 222. For example, forming surface 640 may be positioned at the second end portion 624 of blind hole 620, e.g., such that forming surface 640 is disposed within wall 600 and faces towards first surface 612. Thus, forming surface 640 may form a bottom wall or floor of blind hole 620. Moreover, in certain example embodiments, central axis X of blind hole 620 may intersect a centroid of forming surface 640. In the example embodiment shown in FIGS. 10 and 11, forming surface 640 has a semispherical shape. Such shaping may advantageously assist in forming node 222 with a generally cylindrical shape while also forming nodes 222 with advantageous properties, such less bulk loss at cap 252. The semispherical forming surface 640 may support node 222, e.g., cap 252 of node 222, and also redirect or reflect the pressurized fluid stream 542, as described above, and which may result in improved wall thickness TW of walls 254 of nodes 222.

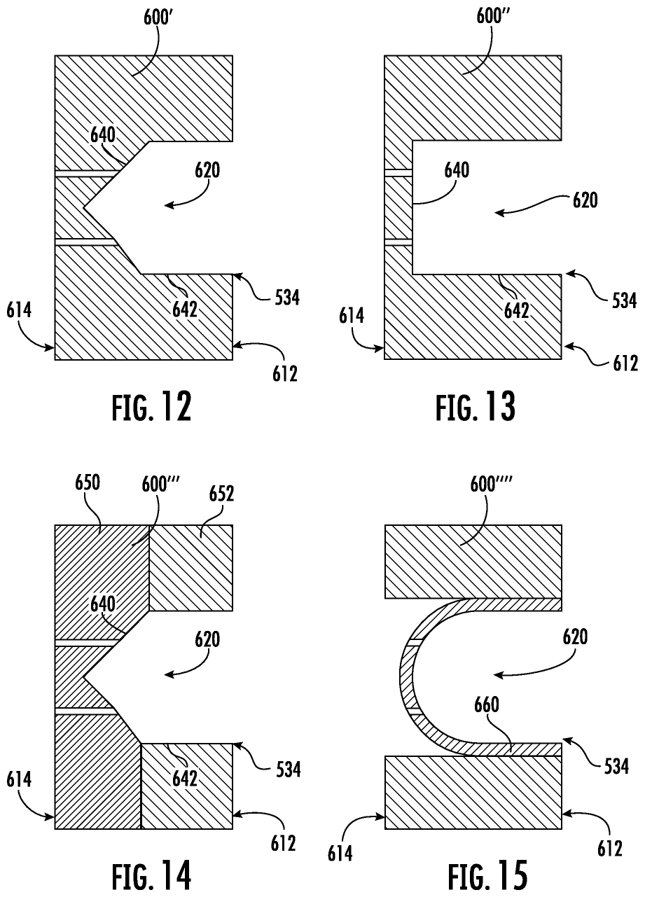

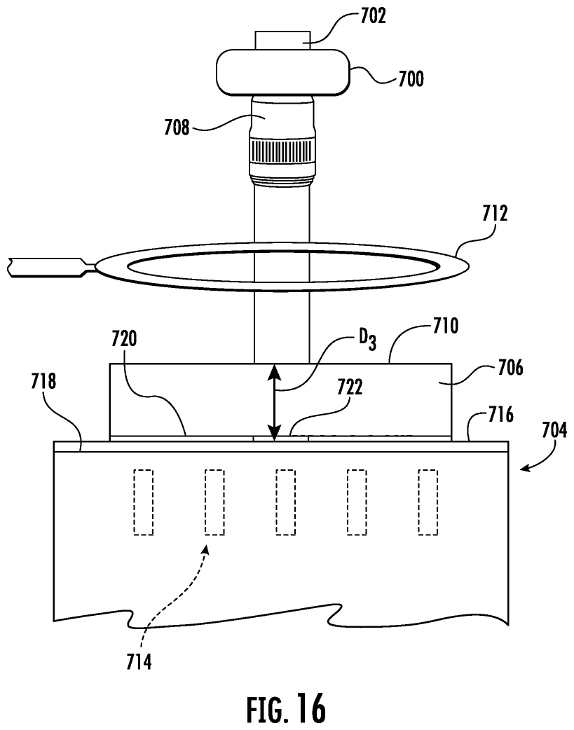

It will be understood that forming surface 640 may have other shapes in other example embodiments. For instance, as shown in FIG. 12, forming surface 640 may have a conical shape. Such shaping may advantageously assist within forming node 222 with a generally cylindrical shape while also forming nodes 222 with advantageous properties, such less bulk loss at cap 252 and improved wall thickness TW of walls 254 of nodes 222. The conical forming surface 640 may support node 222, e.g., cap 252 of node 222, and also redirect or reflect the pressurized fluid stream 542. As another example, forming surface 640 may be planar as shown in FIG. 13. Other shapes for forming surface 640 are also within the scope of the present disclosure.

Turning back to FIG. 10, wall 600 may also define a connecting surface 642, e.g., that extends from first surface 612 to forming surface 640 within formation hole 534. In the example embodiment shown in FIG. 10, connecting surface 642 has a cylindrical shape. Such shaping may advantageously assist within forming node 222 with a generally cylindrical shape while also forming nodes 222 with advantageous properties, such less bulk loss at cap 252 and improved wall thickness TW of walls 254 of nodes 222. Other shapes for connecting surface 642 are also within the scope of the present disclosure. In example embodiments, the shape of connecting surface 642, e.g., along the central axis X, may be constant. Thus, e.g., a cross-sectional area of blind hole 620, e.g., in a plane perpendicular to the central axis X, may be substantially constant at the connecting surface 642. Conversely, the cross-sectional area of blind hole 620, e.g., in a plane perpendicular to the central axis X, may decrease towards the second end portion 624 of blind hole 620 and/or at forming surface 640. Moreover, the cross-sectional area of blind hole 620, e.g., in a plane perpendicular to the central axis X, may be substantially constant from the first end portion 622 of blind hole 620 to at least a middle portion of blind hole 620, e.g., positioned equidistant between first and second end portions 622, 624 of blind hole 620. Thus, connecting surface 642 may be oriented perpendicular to first surface 612 at least to the middle portion of blind hole 620 and/or to the second end portion 624 of blind hole 620 at the forming surface 640.

Forming hole 534 may be formed in wall 600 by drilling, casting, molding, punching, stamping, machining, laser-cutting, water jet cutting, and 3D printing, or any other suitable methodology. Moreover, blind hole 620 may be formed by drilling into wall 600, and the tip of a drill bit may form forming surface 640 within wall 600. As another example, wall 600 may be a multi-piece assembly, and forming holes 534 may be formed by at least two separate pieces of material. Thus, e.g., as shown in FIG. 14, wall 600 may include a first wall section 650 and a second wall section 652 that are mounted to each other to form forming hole 534. Blind hole 620 may be defined in first wall section 650, and outlet passages 630 may be defined in second wall section 652. Moreover, blind hole 620 may be formed by drilling first wall section 650, and outlet passages 630 may be formed by drilling second wall section 652. As yet another example, wall 600 may include an insert 660. Insert 660 may be shaped to form blind hole 620 and may also form outlet passages 630. Insert 660 may be press-fit, adhered, welded, or otherwise suitable mounted within wall 600 to form forming hole 534. Other constructions for forming hole 534 and combinations of the above recited constructions may also be used in other example embodiments.

Turning back to FIG. 5, the process and apparatus 500 may also include one or more fluid entangling devices 540. The most common fluid used in this regard is referred to as spunlace or hydroentangling technology, which uses pressurized water as the fluid for entanglement. As such, the fluid entangling device 540 may include a plurality of high-pressure fluid jets (not shown) to emit a plurality of pressurized fluid streams 542. These fluid streams 542, which are preferably water, may be directed towards the precursor web 510 on the forming surface 530 and can cause the fibers to be further entangled within nonwoven material 200 and/or the precursor forming surface 516 (in the case the precursor forming surface is an underlying web of material). The fluid streams 542 can also cause the fibers in the precursor web 510 to be directed into the forming holes 534 and out of the base plane 241 of the first surface 240 of the nonwoven material 200 and into the transverse direction TT perpendicular to the base plane 241 to form the nodes 222 in the nonwoven material 200 (see FIGS. 2, 3, and 4). The fluid streams 542 can also provide for at least a majority of the plurality of nodes 222 to be configured such that the nodes 222 have an anisotropy value greater than one (1.0), as previously discussed above.

In FIG. 5, a single fluid entangling device 540 is shown, however, depending on the level of entanglement needed and the particular dimensions and qualities of the nonwoven material 200 desired, a plurality of such fluid entangling devices 540 may be used. The entangling fluid streams 542 of the fluid entangling devices 540 may emanate from injectors via jet packs or strips (not shown) consisting of a row or rows of pressurized fluid jets with small apertures of a diameter usually between eight-tenths of a millimeter (0.08) and fifteen-tenths of a millimeter (0.15 mm) and spacing of around a half millimeter (0.5 mm) in the cross-machine direction. The pressure in the jets may be between about five (5) bar and about four hundred (400) bar but typically is less than two hundred (200) bar except for heavy nonwoven materials 10 and when fibrillation is required. Other jet sizes, spacings, numbers of jets and jet pressures may be used depending upon the particular end application. Such fluid entangling devices 540 are well known to those of ordinary skill in the art and are readily available from such manufacturers as Fleissner of Germany and Andritz-Perfojet of France.

The fluid entangling devices 540 may have the jet orifices positioned or spaced between about five millimeters (5 mm) and about twenty millimeters (20 mm), and more typically between about five millimeters (5) and about ten millimeters (10 mm) from the forming surface 530 though the actual spacing can vary depending on the basis weights of the materials being acted upon, the fluid pressure, the number of individual jets being used, the amount of vacuum being used via the fluid removal system 538 and the speed at which the equipment is being run.

In the embodiment shown in FIG. 5, the fluid entangling device 540 is a conventional hydroentangling device the construction and operation of which is well known to those of ordinary skill in the art, such as, for example, U.S. Pat. No. 3,485,706 to Evans, the contents of which is incorporated herein by reference in its entirety for all purposes. Also see the description of the hydraulic entanglement equipment described by Honeycomb Systems, Inc., Biddeford, Me., in the article entitled “Rotary Hydraulic Entanglement of Nonwovens”, reprinted from INSIGHT '86 INTERNATIONAL ADVANCED FORMING/BONDING Conference, the contents of which is incorporated herein by reference in its entirety for all purposes.

The speed of the rotation of the drive roll 520 and the texturizing drum 532 may be set at various speeds with respect to each other. In some example embodiments, the speed of the rotation of the drive roll 520 and the texturizing drum 532 may be the same. In other example embodiments, the speed of the rotation of the drive roll 520 and the texturizing drum 532 may be different. For example, in some example embodiments, the speed of the texturizing drum 532 may be less than the speed of the drive roll 520 to provide for overfeeding of the precursor web 510 on the forming surface 530 on the texturizing drum 532. Such overfeeding may be used to provide varied properties in the nonwoven material 200, such as improved formation of nodes 222 in the nonwoven material 200 and increased height of the nodes 222.

After the fluid entanglement occurs from the fluid entangling streams 542 by the fluid entanglement device 540, the precursor web 510 becomes a hydroentangled web forming the nonwoven material 200 described above that includes a plurality of nodes 222, e.g., with ligaments 223 interconnecting the nodes 222 and/or apertures 221 as described above. The process and apparatus 500 may further include removing the hydroentangled web of nonwoven material 200 from the forming surface 530 and drying the hydroentangled web to provide a three-dimensional nonwoven material 200. Drying of the nonwoven material 200 may occur through known techniques by one of ordinary skill in the art. In example embodiments where the precursory web includes binder fibers, the drying of the nonwoven material 200 may activate the binder fibers. Activating the binder fibers can assist with the preservation of the three-dimensionality of the nonwoven material 200 by helping to preserve the geometry and height of the nodes 222 that extend away from the base plane 241 on the first surface 240 of the nonwoven material 200 (as depicted in FIGS. 2 through 4).

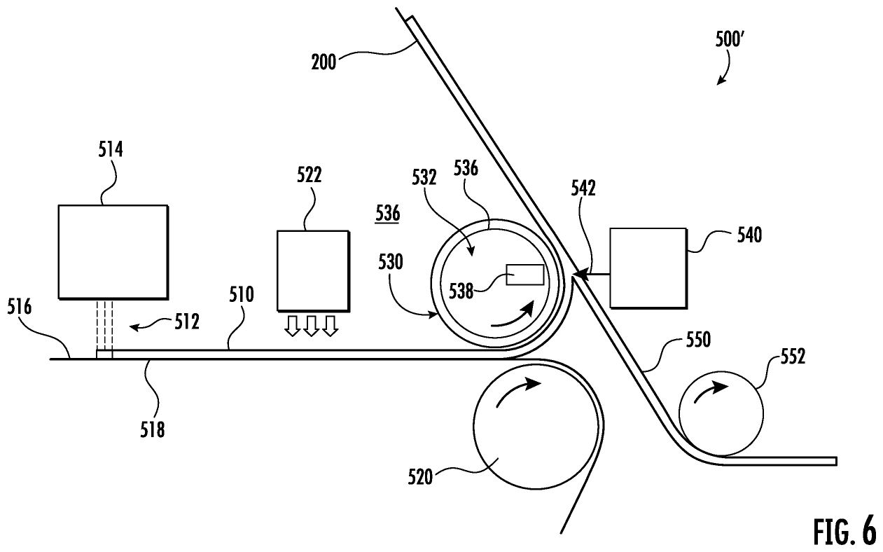

FIG. 6 provides an alternative configuration of the process and apparatus 500′ for manufacturing the nonwoven material 200 as described herein. In FIG. 6, the apparatus and method 100″ may include a support web 550 that is brought into contact with the precursor web 510 prior to the fluid-entangling unit 540. By separating the precursor web 510 from the support web 550, different feeding options of the precursor web 510 and the support web 550 may be achieved. For example, the precursor web 510 may be overfed to the fluid-entangling unit 540 through sizes and speeds of drive roll 520 in comparison to the texturizing drum 532, whereas the support web 550 may be supplied to the fluid-entangling unit 540 at a match speed of the texturizing drum 532 through drive roll 552. This is further described in U.S. Pat. No. 9,474,660 invented by Kirby, Scott S. C. et al., which is incorporated herein in its entirety to the extent not contradictory herewith.

As also depicted in FIG. 7, in some example embodiments, the nonwoven material 200 may be combined with an additional web, such as a carrier material 560. The carrier material 560 can be coupled to the nonwoven material 200 through any suitable coupling mechanism, such as by adhesive bonding or mechanical bonding, for example, ultrasonic bonding, pressure bonding, thermal bonding, or any other suitable bonding mechanism. The carrier material 560 may be coupled to the nonwoven material 200 after the fluid-entangling unit 540. In some example embodiments, the carrier material 560 may be coupled to the nonwoven material 200 after the nonwoven material 200 is dried. In other example embodiments, the carrier material 560 may be coupled to the nonwoven material 200 before the nonwoven material 200 is dried. The carrier material 560 may provide additional tensile strength to the nonwoven material 200 and may improve handling in high-speed converting and/or manufacturing environments. The carrier material 560 may be a liquid permeable material and be coupled to the nonwoven material 200 such that the carrier material 560 adjoins the first surface 240 of the nonwoven material 200 including the nodes 222. It is also noted that a carrier material 560 could be added to the process and apparatus 500″ as depicted and described with respect to FIG. 6, in which the support web 550 is supplied to the fluid entangling unit 540 separate from the precursor web 510.

Although FIGS. 5 through 7 depict example processes and apparatuses 500, 500′, and 500″ of fluid entanglement for manufacturing the nonwoven material 200, it is contemplated that variances from these processes and apparatuses 500, 500′, and 500″ may be used. For example, as mentioned previously, the precursor web 510 may be provided utilizing various techniques other than a wet-laying process, such as being formed by a foam-laying process or a carding process. Additionally, the precursor web 510 may be provided on a separate line and wound onto core rolls (not shown) and then transported to a separate manufacturing line to engage in the fluid entanglement process by a fluid entangling device 540 as discussed above.

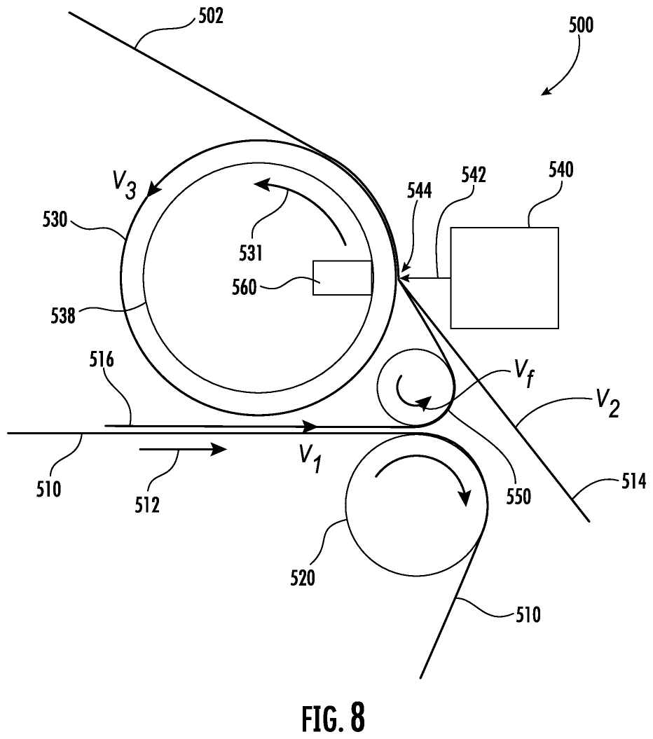

FIG. 8 depicts a further an apparatus 500″ and associated process that may be particularly suited to aspects of the present disclosure. In some example embodiments, the apparatus 500″ and associated process may be the same or similar to those described with respect to the processes and apparatuses in U.S. Pat. No. 11,365,495, titled “Process for making fluid-entangled laminate webs with hollow projections and apertures”, to Mark J. Beitz et al., the entirety of which is hereby incorporated by reference.

Apparatus and process 500″ may include entangling and hence bonding a support web 513 and a precursor web 510, sometimes called a projection web 510. In some example embodiments, the fibers of the support web 513 may be similar to those described with respect to the support web 14 in U.S. Pat. No. 9,327,473 to Scott Kirby et al. Similarly, the fibers of the projection web 510 may be similar to those described with respect to the projection web 16 in U.S. Pat. No. 9,327,473.

The apparatus 500′″ and associated process can include a transport belt 518, a transport belt drive roll 520, a projection forming surface 530, a fluid entangling device 540, an optional overfeed roll 551, and a fluid removal system 538, such as a vacuum or other conventional suction device. Such vacuum devices and other mechanisms are well known to those of ordinary skill in the art. The transport belt 510 is used to carry the projection web 510 into the apparatus 500″. If any pre-entangling or wet-forming of the projection web 510 is to be done on the projection web 510 upstream of the process 500″ shown in FIG. 8, the transport belt 518 may be porous (for example, as shown FIGS. 5-7). The transport belt 518 travels in a first direction (which is the machine direction) as shown by arrow 512 at a first speed or velocity V₁. The transport belt 518 may be driven by the transport belt drive roller 520 or other suitable mechanism as are well known to those of ordinary skill in the art.

The projection forming surface 530 moves in the machine direction as shown by arrow 531 in FIG. 8 at a speed or velocity V₃. The projection forming surface 530 is driven and speed controlled by any suitable drive mechanism (not shown), such as electric motors and gearing as are well known to those of ordinary skill in the art. The projection web 510 is fed into the apparatus and process 500″ at a speed V₁, the support layer 513 is fed into the apparatus and process 500″ at a speed V₂, and the fluid-entangled laminate web 200 exits the apparatus and process 500′″ at a speed V₃, which is the speed of the projection forming surface 530 and may also be referred to as the projection forming surface speed. As will be explained in greater detail below, these speeds V₁, V₂, and V₃ may be the same as one another or varied to change the formation process and the properties of the resultant fluid-entangled laminate web 200. Feeding both the projection web 510 and the support layer 513 into the process 500″ at the same speed (V₁ and V₂) may produce a fluid-entangled laminate web 200 according to example aspects of the present disclosure with the desired nodes 222. Feeding both the projection web 501 and the support layer 513 into the process at the same speed, which is faster than the machine direction speed (V₃) of the projection forming surface 530, may also form the desired nodes 222.

Also shown in FIG. 8 is an optional overfeed roll 551, which may be driven at a speed or rate V_{f}. The overfeed roll 551 may be run at the same speed as the speed V₁ of the projection web 510, in which case V_{f} may equal V₁ or V_{f} may be run at a faster rate to tension the projection web 510 upstream of the overfeed roll 551 when overfeed is desired. Over-feed occurs when one or both of the incoming webs/layers (510, 513) are fed onto the projection forming surface 530 at a greater speed than the projection forming surface speed of the projection forming surface 530. It has been found that improved projection formation in the projection web 510 may be affected by feeding the projection web 510 onto the projection forming surface 530 at a higher rate than the incoming speed V₂ of the support layer 513. In addition, however, it has been discovered that improved properties and projection formation may be accomplished by varying the feed rates of the webs/layers (510, 513) and by also using the overfeed roll 551 just upstream of the texturizing drum 530 to supply a greater amount of fiber via the projection web 510 for subsequent movement by the entangling fluid 542 down into the forming holes 534 in the texturizing drum 530. In particular, by overfeeding the projection web 510 onto the texturizing drum 530, improved projection formation may be achieved including increased projection height.

In order to provide an excess of fiber so that the height of the nodes 222 may be increased or maximized, the projection web 510 may be fed onto the texturing drum 530 at a greater surface speed (V₁) than the texturizing drum 530 is traveling (V₃). Referring to FIG. 8, when overfeed is desired, the projection web 510 is fed onto the texturizing drum 530 at a speed V₁ while the support layer 513 is fed in at a speed V₂ and the texturizing drum 530 is traveling at a speed V₃, which is slower than V₁ and may be equal to V₂. The overfeed percent or ratio, the ratio at which the projection web 510 is fed onto the texturizing drum 530, may be defined as OF=[(V₁/V₃)−1]×100 where V₁ is the input speed of the projection web 510 and V₃ is the output speed of the resultant fluid-entangled laminate web 200 and the speed of the texturizing drum 530. (When the overfeed roll 550 is being used to increase the speed of the incoming material onto the texturizing drum 530, it should be noted that the speed V₁ of the material after the overfeed roll 551 may be faster than the speed V₁ upstream of the overfeed roll 551. In calculating the overfeed ratio, it is this faster speed V_{f} that should be used.) Good formation of the nodes 222 has been found to occur when the overfeed ratio is between about five percent (5%) and about fifty percent (50%). Note too, that this overfeeding technique and ratio may be used with respect to not just the projection web 510 only but to a combination of the projection web 510 and the support layer 513 as the webs 513, 510 are collectively fed onto the projection forming surface 530.

In order to minimize the length of projection web 510 that is supporting its own weight before being subjected to the entangling fluid 542 and to avoid wrinkling and folding of the projection web 510, the overfeed roll 551 may be used to carry the projection web 510 at speed V₁ to a position close to the texturizing zone 544 on the texturizing drum 530. In the example illustrated in FIG. 8, the overfeed roll 551 is driven off the transport belt 518, but it is also possible to drive the overfeed roll 551 separately so as to not put undue stress on the incoming projection web material 510. The support layer 513 may be fed into the texturizing zone 544 separately from the projection web 510 and at a speed V₂ that may be greater than, equal to or less than the texturizing drum speed V₃ and greater than, equal to or less than the projection web 510 speed V₁. Preferably, the support layer 513 is drawn into the texturizing zone 544 by frictional engagement with the projection web 510 positioned on the texturizing drum 530, and, once on the texturizing drum 530, the support layer 513 has a surface speed close to the speed V₃ of the texturizing drum 530, or the support layer 513 may be positively fed into the texturizing zone 544 at a speed close to the texturizing drum speed of V₃. The texturizing process causes some contraction of the support layer 513 in the machine direction 531. The overfeed of either the support layer 513 or the projection web 510 may be adjusted according to the particular materials and the equipment and conditions being used so that the excess material that is fed into the texturizing zone 544 is used up thereby avoiding any unsightly wrinkling in the resultant fluid-entangled laminate web 200. As a result, the two webs/layers (510, 513) will usually be under some tension at all times despite the overfeeding process. The take-off speed of the fluid-entangled laminate web 200 must be arranged to be to be close to the texturizing drum speed V₃ such that excessive tension is not applied to the fluid-entangled laminate web 200 in removal of the laminate 200 from the texturizing drum 530 as such excessive tension would be detrimental to the clarity and size of the nodes 222.

After the fluid entanglement occurs from the fluid entangling streams 542 by the fluid entanglement device 540, the projection web 510 and the support web 513 become a laminate nonwoven web forming the nonwoven material 200 described above that includes nodes 222, ligaments 223 interconnecting the nodes 222, and/or apertures 221 as described above. Such nonwoven materials 200 may be devoid of any binders—such as adhesive or the like—and may be held together solely through fiber entanglement. The process and apparatus 500′″ may further include removing the web of nonwoven material 200 from the forming surface 530 and drying the hydroentangled web to provide a three-dimensional nonwoven material 200. Drying of the nonwoven material 200 may occur through known techniques by one of ordinary skill in the art. In example embodiments where the precursory web includes binder fibers, the drying of the nonwoven material 200 may activate the binder fibers. Activating the binder fibers can assist with the preservation of the three-dimensionality of the nonwoven material 200 by helping to preserve the geometry and height of nodes 222 that extend away from the base plane 241 on the first surface 240 of the nonwoven material 200 (as depicted in FIGS. 3 and 4).

Suitable materials for the projection web 510 can be similar to those described above with respect to precursor web 510 of apparatuses and methods 500, 500′, and 500″. Suitable materials for the support layer 513 can include a fibrous nonwoven web made from a plurality of randomly deposited fibers which may be staple length fibers such as are used, for example, in carded webs, air laid webs, etc. Alternatively, the materials may be more continuous fibers such as are found in, for example, meltblown or spunbond webs. Due to the functions the support layer 513 must perform, the support layer 513 may have a higher degree of integrity than the projection web 510. In this regard, the support layer 513 may be able to remain substantially intact when the support layer 513 is subjected to the fluid-entangling process discussed in greater detail below. The degree of integrity of the support layer 513 may be such that the material forming the support layer 513 resists being driven down into and filling the nodes 222 of the projection web 510. As a result, when the support layer 513 is a fibrous nonwoven web, it may be desirable that the support layer 513 has a higher degree of fiber-to-fiber bonding and/or fiber entanglement than the fibers in the projection web 510. While it is desirable to have fibers from the support layer 513 entangle with the fibers of the projection web 510 adjacent the interface between the two layers, it is generally desired that the fibers of this support layer 513 not be integrated or entangled into the projection web 510 to such a degree that large portions of these fibers find their way inside the nodes 222.

As noted above, nodes 222 of nonwoven material 200 may have improved properties relative to known nonwoven materials—particularly after being wound and unwound from rolls for transport and/or after being placed onto an absorbent article and compressed within a packaging. For instance, one example beneficial property of the nonwoven material 200 may include fiber orientation. In example embodiments, such as referenced below with respect to Example B, at least a majority of nodes 222 may be configured such that the nodes 222 have an anisotropy value greater than ninety-two hundredths (0.92) as measured by the Node Analysis Test Method, described in the Test Methods section herein. The nodes 222 may thus have a higher level of fiber alignment in a transverse direction TT perpendicular to the base plane 241 on the first surface 240 of the nonwoven material 200. A nonwoven material with nodes formed in cylindrical holes such that the distal end portion of the nodes do not contact any surface—e.g., un-socketed holes—was provided as a comparative example, Example A. The anisotropy values for the nonwoven material made according to example aspects of the present disclosure (formed with the socketed formation holes 534) and the comparative example (formed without the socketed formation holes 534) are shown below. The basis weight for both examples was 70 gsm.

The comparative example A material and the example B material were both formed according to the apparatus and process described with respect to FIG. 8 and apparatus 500″. The forming surface description is indicated in TABLE 1 below. The comparative example A material was formed with cylindrical hole forming holes 534 having the indicated depth and diameter, which extended through the entire thickness of the forming surface. The example B material was formed with forming holes 534 having a cylindrical diameters and forming blind holes having a semispherical shape at the second end 642 of the forming surface 640 defining the forming holes 534. Both materials A and B were formed with a support layer comprised of a spunbond material having a basis weight of 17 gsm. The spunbond material was formed of polypropylene fibers having a denier of 1.8. The projection layers of the materials A and B comprised carded webs having a basis weight of 53 gsm. The carded webs comprised polyester fibers having a denier of 1.3.

TABLE 1

| | | | | Material Properties | | | |
|---|---|---|---|---|---|---|---|
| | | | | | Average | | |
| | Forming surface | | | Average | Node | Average | |
| | Hole | Hole | | Node | Wall | Node Cap | Average |
| | Depth | Diameter | Type | Height | Thickness | Thickness | Anisotropy |
| Comparative | 4.7 mm | 4.7 mm | Through | 1446 μm | 422.5 μm | 418.8 μm | 0.91 |
| Example A | | | Hole | | | | |
| Example B | 3.7 mm | 4.7 mm | Blind | 1307 μm | 453.3 μm | 370.9 μm | 0.97 |
| | | | Hole | | | | |

As shown in the above Table 1, the nonwoven material made according to example aspects of the present disclosure (formed with the socketed formation holes 534—Example B) included an anisotropy value greater than 0.92 (e.g., and less than 1.1), namely having an anisotropy values of 0.97. Other exemplary nonwoven material of the present disclosure may have anisotropy values of between 0.92 and 0.97, or even greater than 0.97, such as up to 1.0 or 1.1. Such values may be achieved by adjusting the specific fiber properties of the precursor web 510 (e.g., fiber length, fiber denier, etc.) as well as the fluid pressures and nozzle spacing.

Not to be bound by theory, but it is believed that the improved anisotropy values in the nodes 222 of the nonwoven material 200 described herein can be created due to the blind hole 620 supporting node 222 and also redirecting or reflecting the pressurized fluid stream 542 to avoid bulk loss and improved wall thickness TW of walls 254 of nodes 222.

Additionally, it is believed that the increased anisotropy values of the nonwoven materials 200 according to this description provide improved compression resistance for the nonwoven material 200 as compared to other nonwoven materials. With improved compression resistance, the nonwoven material 200 can maintain loft through application and use in a variety of environment where the nonwoven material 200 may be exposed to compressive forces. For example, when used in an absorbent article, the nonwoven material 200 can be under compressive forces from an initial packaging state of being in compressed packaging to application on the wearer if the wearer is in a sitting or lying position on the absorbent article. By providing improved resistance to compression, the nonwoven material 200 can help maintain void volume for accepting, transferring, and/or storing body exudates from a wearer. In doing so, the nonwoven material 200 can provide enhanced skin benefits for the wearer by helping keep body exudates away from a wearer's skin and potential product improvements by keeping body exudates away from the edges of the absorbent article, which may be a source of leaks.

The Compression Linearity Test, as described fully in the Test Methods section herein, is designed to measure the compression properties of the nonwoven material by compressing the material at a constant rate between two plungers until reaching a maximum preset force. The displacement of the top plunger compressing the material is detected by a potentiometer. The amount of pressure taken to compress the sample (P, gf/cm²) vs. thickness (displacement) of the material (T, mm) is plotted. The value of compression linearity represents the degree of linearity of the compression curve. The higher the compression linearity value, the more resistant a material is to being compressed. The compression linearity for the nonwoven material made according to example aspects of the present disclosure (formed with the socketed formation holes 534) and the comparative example (formed without the socketed formation holes 534) are shown below.

TABLE 2

| | | | | Material Properties | | | |
|---|---|---|---|---|---|---|---|
| | | | | | Average | | |
| | Forming surface | | | Average | Node | Average | |
| | Hole | Hole | | Node | Wall | Node Cap | Compression |
| | Depth | Diameter | Type | Height | Thickness | Thickness | Linearity |
| Comparative | 4.7 mm | 4.7 mm | Through | 1446 μm | 422.5 μm | 418.8 μm | 0.849 |
| Example A | | | Hole | | | | |
| Example B | 3.7 mm | 4.7 mm | Blind | 1307 μm | 453.3 μm | 370.9 μm | 1.03 |
| | | | Hole | | | | |

The nonwoven material made according to example aspects of the present disclosure (formed with the socketed formation holes 534) exhibited a compression linearity of about 1.03 whereas the comparative example (formed without the socketed formation holes 534) exhibited a compression linearity of less than 0.849. Thus, in example embodiments, the nonwoven material 200 may have a compression linearity of greater than 0.90, more preferably greater than 0.95, more preferably greater than 0.975, even more preferably greater than 0.99, and most preferably greater than 1.0. In some example embodiments, the nonwoven material 200 may have a compression linearity of between about 0.9 and 1.2, such as between about 0.9 and 1.1, such as between about 0.9 and 1.05, such as between about 0.9 and 1.03, such as between about 0.95 and about 1.1, such as between about 0.95 and 1.05.

As noted above, apertures 221 and nodes 222 may enhance the ability of a material, such as nonwoven material 200, to intake and distribute BM material (also referred to herein as feces or fecal matter), resulting in less pooling of the BM on the nonwoven material 200 and therefore less BM disposed against a skin of a wearer of an absorbent article including the nonwoven material 200. In order to determine the ability of different nonwoven materials to effectively handle simulated BM, a nonwoven material (Material B), according to example aspects of the present disclosure, was tested utilizing a test method which determined a BM pooled percent value. Such a test method is described as a “Determination of Residual Fecal Material Simulant” test method in U.S. Pat. No. 9,480,609, titled “Absorbent Article”, the entirety of which is hereby incorporated by reference to the extent not contradictory herewith. A nonwoven material with nodes formed in cylindrical holes such that the distal end portion of the nodes do not contact any surface was provided as a first comparative example (Comparative Material A), and a three-dimensional topical intake material from a commercial Huggies® Special Delivery diaper product including nodes protruding from a base-plane of the material but without any apertures was provided as a second comparative example.

| | BM Pooled | BM Pooled |
|---|---|---|
| | on Mannequin | on Liner |
| Comparative Material A | 0.51 g | 2.62 g |
| Huggies ® Special Delivery | 0.57 g | 2.21 g |
| Material B | 0.37 g | 1.46 g |

Primarily, it can be seen how effective nonwoven material 200 with apertures 221 and nodes 222 are in terms of reducing the amount of pooled BM on such materials. For example, as shown in the above Table 2, Material B, formed with the socketed formation holes 534, performed better than both the Material A (formed without the socketed formation holes 534) and the Huggies® Special Delivery material in terms of an amount of BM left pooled. Thus, in example embodiments, the amount of residual fecal material simulant remaining on the nonwoven material 200 following insult with fecal material simulant according to the Determination of Residual Fecal Material Simulant Test Method may be less than about 2.0 grams, such as less than 1.9 grams, such as less than 1.8 grams, such as less than 1.7 grams, such as less than 1.6 grams, such as less than 1.5 grams, such as less than 1.47 grams. In any of these examples, the amount of residual fecal material simulant remaining on the nonwoven material 200 following insult with fecal material simulant according to the Determination of Residual Fecal Material Simulant Test Method may be greater than or equal to about 1.46 grams, such as greater than or equal to about 1.5 grams, such as greater than or equal to about 1.6 grams, such as greater than or equal to about 1.7 grams, such as greater than or equal to about 1.8 grams, such as greater than or equal to about 1.9 grams.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

### Test Methods

The test methods detailed herein and the measured values were performed on previously compressed material. For example, the prior to any measurements, the sample materials were selected from materials that had been either wound and unwound from rolls of material—as is typical for commercial manufacture and transport of nonwoven web materials-removed from commercial product that had been disposed within compressed packaging.

### Node Analysis Test Method

The anisotropy of fibers in the nodes 222 can be determined by using the image analysis measurement method described herein. This test method can also measure node height as well as the percentage of fibers and voids within a node 222.

In this context, fiber anisotropy is considered for a plurality of nodes 222 from each respective material. Generally, the image analysis method determines a numeric value of anisotropy from a cross-sectional image of a node 222 via a specific image analysis measurement parameter named anisotropy. The anisotropy of a node 222 can be measured by using x-ray Micro-computed Tomography (a.k.a. Micro-CT) to non-destructively acquire images with subsequent image analysis techniques to detect fiber components and then measuring the anisotropy of said components within the node 222 regions only. The image analysis algorithm performs detection, image processing and measurement and also transmits data digitally to a spreadsheet database. The resulting measurement data are used to compare the anisotropy of differing structures possessing fibrous node 222 components.

The method for determining the anisotropy in each structures nodes' fibers includes the first step of acquiring digital x-ray Micro-CT images of a sample. These images are acquired using a SkyScan 1272 Micro-CT system available from Bruker microCT (2550 Kontich, Belgium). The sample is attached to a mounting apparatus, supplied by Bruker with the SkyScan 1272 system, so that it will not move under its own weight during the scanning process. The following SkyScan 1272 conditions are used during the scanning process:

Camera Pixel Size (um) = 9.0
Source Voltage (kV) = 35
Source Current (uA) = 225
Image Pixel Size (um) = 6.0
Image Format = TIFF
Depth (bits) = 16
Rotation Step (deg.) = 0.10
Use 360 Rotation = NO
Frame Averaging = ON (6)
Random Movement = ON (1)
Flat Field Correction = ON
Filter = No Filter

After sample scanning is completed, the resulting image set is then reconstructed using the NRecon program provided with the SkyScan 1272 Micro-CT system. While reconstruction parameters can be somewhat sample dependent, and should be known to those skilled in the art, the following parameters should provide a basic guideline to an analyst:

Image File Type = JPG
Pixel Size (um) = 6.00
Smoothing = 1 (Gaussian)
Ring Artifact Correction = 10
Beam Hardening Correction (%) = 10

After reconstruction is completed, the resulting image data set is now ready for extraction of cross-sectional image slices using the Bruker SkyScan software package called DataViewer. After downloading the entire reconstructed image data set into DataViewer, the analyst, skilled in the art of Micro-CT technologies, must then select and extract cross-sectional image slices which reside at or near the center of nodes present in each respective sample. One centered node 222 should be obtained for each image selected. For a typical specimen, this process will result in 4-6 images and from which 4-6 nodes 222 will be available for analysis. The analyst should then re-number the images sequentially (e.g. 1, 2, 3, etc.) by changing the image file suffix numbers.

Once a set of cross-sectional Micro-CT images have been acquired and re-numbered from each specimen, anisotropy measurements can now be made using image analysis software.

The image analysis software platform used to perform the anisotropy measurements is a QWIN Pro (Version 3.5.1) available from Leica Microsystems, having an office in Heerbrugg, Switzerland.

Thus, the method for determining the anisotropy of a given sample includes the step of performing several anisotropy measurements on the Micro-CT image set. Specifically, an image analysis algorithm is used to read and process images as well as perform measurements using Quantimet User Interactive Programming System (QUIPS) language. The image analysis algorithm is reproduced below.

```
DEFINE VARIABLES & OPEN FILES
  The following line designates the computer location where data is sent to
  Open File  (C:\Data\94054 - Nhan (patent)\z-micro-ct data.xls, channel #1)
  PauseText  (“Enter the number of the final image in the set.”)
  Input  (IMAGES)
  SAMPLE ID AND SET UP
  Enter Results Header
  File Results Header (channel #1)
  File Line  (channel #1)
  Measure frame  (x 31, y 61, Width 1737, Height 793)
  Image frame  (x 0, y 0, Width 1768, Height 854 )
    -- Calvalue 6.0 um/pixel
  CALVALUE = 6.0
  Calibrate  (CALVALUE CALUNITS$ per pixel)
    -- Enter image prefix name of set of images to analyze
  PauseText  ( “Enter image file prefix name.” )
  Input  ( TITLE$ )
  File  ( “Rep. #”, channel #1 )
  File  ( “% Fiber”, channel #1 )
  File  ( “% Voids”, channel #1 )
  File  ( “Height (um)”, channel #1 )
  File  ( “Anisotropy”, channel #1 )
  File Line  ( channel #1 )
  For  ( REPLICATE = 1 to IMAGES, step 1 )
    Clear Accepts
    IMAGE ACQUISITION AND DETECTION
    ACQOUTPUT = 0
The following two lines indicate the computer location of the Micro-CT images to be read during the
image analysis process.
    ACQFILE$ = “C:\Images\94054 - Nhan\Z-slices\”+TITLE$+“”+STR$(REPLICATE)+“.jpg”
    Read image  ( from file ACQFILE$ into ACQOUTPUT )
    Colour Transform  ( Mono Mode )
    Grey Transform  ( WSharpen from Image0 to Image1, cycles 3, operator Disc )
    Detect  ( whiter than 64, from Image1 into Binary0 )
    IMAGE PROCESSING
    PauseText  ( “Select region of interest for analysis.” )
    Binary Edit [PAUSE] ( Accept from Binary0 to Binary1, nib Fill, width 2 )
    Binary Amend  ( Close from Binary1 to Binary2, cycles 30, operator Disc, edge erode on )
    Binary Identify  ( FillHoles from Binary2 to Binary3 )
    Binary Amend  ( Open from Binary3 to Binary4, cycles 40, operator Disc, edge erode on )
    PauseText  ( “Clean up any over extended ROI areas.” )
    Binary Edit [PAUSE] ( Reject from Binary4 to Binary5, nib Fill, width 2 )
    PauseText  ( “Draw vertical line thru the thickest region binary.” )
    Binary Edit [PAUSE] ( Accept from Binary5 to Binary7, nib Rect, width 2 )
    Binary Logical  ( C = A AND B : C Binary6, A Binary1, B Binary5 )
        MEASURE ANALYSIS REGIONS
      -- Analysis Region Fiber Area
    MFLDIMAGE = 6
    Measure field  ( plane MFLDIMAGE, into FLDRESULTS(2), statistics into FLDSTATS(7,2) )
          Selected parameters: Area, Anisotropy
    FIBERAREA = FLDRESULTS(1)
    ANISOTROPY = FLDRESULTS(2)
      -- Analysis Region Area
    MFLDIMAGE = 5
    Measure field  ( plane MFLDIMAGE, into FLDRESULTS(1), statistics into FLDSTATS(7,1) )
          Selected parameters:  Area
    ROIAREA = FLDRESULTS(1)
    PERCFIBER = FIBERAREA/ROIAREA*100
    PERCVOIDS = 100-PERCFIBER
      -- Measure Node Height
    Measure feature  ( plane Binary7, 8 ferets, minimum area: 24, grey image: Image0 )
          Selected parameters: X FCP, Y FCP, Length
    LENGTH = Field Sum of ( PLENGTH(FTR) )
        OUTPUT DATA
    File  ( REPLICATE, channel #1, 0 digits after “.” )
    File  ( PERCFIBER, channel #1, 1 digit after “.” )
    File  ( PERCVOIDS, channel #1, 1 digit after “.” )
    File  ( LENGTH, channel #1, 1 digit after “.” )
    File  ( ANISOTROPY, channel #1, 2 digits after “.” )
    File Line  ( channel #1 )
  Next  ( REPLICATE )
  Close File  (channel #1)
  END
```

The QUIPS algorithm is executed using the QWIN Pro software platform. The analyst is initially prompted to enter the number of images in the set for a particular specimen. Next, the analyst is prompted to enter specimen identification information which is sent to the EXCEL file.

The analyst is next prompted by an interactive command window and an input window to enter the image file prefix of the Micro-CT images to be analyzed. After this step, all subsequent images for a given sample will be read automatically by the image analysis algorithm described above.

The analyst is next prompted to manually select, with the computer mouse, the node region of interest for analysis. Care should be taken to select the entire node so as to include the tapered sections just down to base plane 241 of the material 200.

After several steps of image processing that will occur automatically, the analyst will again be prompted to clean up any over-extended region of interest (ROI) areas. This is done using the computer mouse as well as toggling the overlying binary image on and off by simultaneously using the ‘control’ and ‘b’ keys on the computer keyboard. After this step, the binary should only be covering the node.

Lastly, the analyst will be prompted to use the computer mouse to draw a vertical line thru the tallest region of the binary image. This line will be used by the computer algorithm to measure the height of the node 222.

The process of selecting the node 222 region of interest, clean up of over-extended regions, and drawing a vertical line thru the tallest region of the node 222 will repeat until all the images for a particular specimen have been analyzed.

After all images have been analyzed, the following measurement parameter data will be located in the corresponding EXCEL file:

Replicate #
% Fiber
% Voids
Height
Anisotropy

There will be 4-6 values listed in columns for each of these parameters. For the purposes of comparing anisotropy values between specimens, the data in the column labeled ‘Anisotropy’ can be compared between different specimens by performing a Student's T analysis at the 90% confidence level.

### Material Sample Analysis Test Method

The Material Sample Analysis Test Method as described herein can be used for determining the size of an aperture 221, the roundness of an aperture 221, the aspect ratio for an aperture 221, the two-dimensional area of a node 222, and node 222 density and spacing. This test method involves obtaining two separate digital images of the sample.

### Material Apertured Zone Sample Analysis Set-Up and Determination

An exemplary setup for acquiring the images of the apertured zone is representatively illustrated in FIG. 15. Specifically, a CCD video camera 700 (e.g., a Leica DFC 300 FX video camera available from Leica Microsystems of Heerbrugg, Switzerland) is mounted on a standard support 702, such as a Polaroid MP-4 Land Camera standard support formerly available from Polaroid Resource Center in Cambridge, MS, and now potentially available from a resource such as eBay. The standard support 702 is attached to a macro-viewer 704, such as a KREONITE macro-viewer available from Dunning Photo Equipment, Inc., having an office in Bixby, Oklahoma. An auto stage 706 is placed on the upper surface of the macro-viewer 704. The auto stage 706 is used to automatically move the position of a given sample for viewing by the camera 700. A suitable auto stage 706 is Model H112, available from Prior Scientific Inc., having an office in Rockland, MA.

The specimen (not shown in FIG. 15) is placed on the auto stage 706 of a Leica Microsystems QWIN Pro Image Analysis system, under the optical axis of a sixty-millimeter (60 mm) lens 708 having an f-stop setting of four (4), such as a Nikon AF Micro Nikkor, manufactured by Nikon Corporation, having an office in Tokyo, Japan. The lens 708 is attached to the camera 700 using a c-mount adaptor. The distance from the front face of the lens 708 to the sample is approximately fifty-five centimeters (55 cm). The sample is laid flat on the auto stage surface 710 and any wrinkles removed by gentle stretching and/or fastening it to the auto stage surface 710 using transparent adhesive tape at outer edges of the sample. The sample surface is illuminated with incident fluorescent lighting provided by a sixteen inch (16″) diameter, forty watt (40 w), Circline fluorescent light 712, such as that manufactured by General Electric Company, having an office in Boston, MA. The light 712 is contained in a fixture that is positioned so the light 712 is centered over the sample and is approximately three centimeters (3 cm) above the sample surface. The illumination level of the light 712 is controlled with a Variable Auto-transformer (not shown), type 3PN1010, available from Staco Energy Products Co. having an office in Dayton, OH. Transmitted light is also provided to the sample from beneath the auto stage by a bank of four, two foot (2 ft.), EMC, Double-End Powered LED tube light 714 which are dimmable and available from Fulight Optoelectronic Materials, LLC. The LED light 714 are covered with a diffusing plate 716. The diffusing plate 716 is inset into, and forms a portion of, the upper surface 718 of the macro-viewer 704. This illumination source is overlaid with black mask 720 possessing a 3-inch by 3-inch opening 722. The opening 722 is positioned so that opening 722 is centered under the optical axis of the camera 700 and lens 708 system. The distance D3 from the fluorescent light opening 722 to the surface 710 of the auto stage 706 is approximately seventeen centimeters (17 cm). The illumination level of the fluorescent light bank 714 is also controlled with a separate power control box (not shown) configured for dimmable LED lights.

The image analysis software platform used to perform the percent open area and aperture size measurements is a QWIN Pro (Version 3.5.1) available from Leica Microsystems, having an office in Heerbrugg, Switzerland. Alternatively, LAS Macro Editor, the next generation of software following QWIN Pro, could be used to perform the analysis. The system and images are also accurately calibrated using the QWIN software and a standard ruler with metric markings at least as small as one millimeter. The calibration is performed in the horizontal dimension of the video camera image. Units of millimeters per pixel are used for the calibration.

Thus, the method for determining the percent open area and opening size of a given specimen includes the step of performing measurements on the transmitted light image. Specifically, an image analysis algorithm is used to acquire and process images as well as perform measurements using Quantimet User Interactive Programming System (QUIPS) language. The image analysis algorithm is reproduced below. For purposes of clarity, the references in the algorithm to “bumps” or “projections” refers to nodes 222 for the nonwoven material 200 and the references to “open areas” or “apertures” refers to openings 221 for the nonwoven material 200.

```
DEFINE VARIABLES & OPEN FILES
  The following line designates the computer location where data is sent to
  Open File  (C:\Data\94054 - Nhan (patent)\data.xls, channel #1)
  TOTCOUNT = 0
  TOTFIELDS = 0
  MFRAMEH = 875
  MFRAMEW = 1249
  SAMPLE ID AND SET UP
  Configure  ( Image Store 1392 x 1040, Grey Images 81, Binaries 24 )
  Enter Results Header
  File Results Header  ( channel #1 )
  File Line  ( channel #1 )
  PauseText  ( “Enter sample image prefix name now.” )
  Input  ( TITLE$ )
  PauseText  ( “Set sample into position.” )
  Image Setup DC Twain [PAUSE] ( Camera 1, AutoExposure Off, Gain 0.00, ExposureTime 34.23
  msec, Brightness 0, Lamp 38.83 )
  Measure frame  ( x 74, y 110, Width 1249, Height 875 )
  Image frame  (x 0, y 0, Width 1392, Height 1040 )
  -- Calvalue = 0.0377 mm/px
  CALVALUE = 0.0377
  Calibrate  ( CALVALUE CALUNITS$ per pixel )
  FRMAREA = MFRAMEH*MFRAMEW*(CALVALUE**2)
  Clear Accepts
  For  ( SAMPLE = 1 to 1, step 1 )
    Clear Accepts
    File  ( “Field No.”, channel #1, field width: 9, left justified )
    File  ( “% Open Area”, channel #1, field width: 7, left justified )
    File  ( “Bump Density”, channel #1, field width: 13, left justified )
    File  ( “Bump Spacing”, channel #1, field width: 15, left justified )
    File Line  ( channel #1 )
    Stage  ( Define Origin )
    Stage  ( Scan Pattern, 1 x 5 fields, size 82500.000000 x 39000.000000 )
    IMAGE ACQUISITION I - Projection isolation
    For  ( FIELD = 1 to 5, step 1 )
      Measure frame  ( x 74, y 110, Width 1249, Height 875 )
      Display  ( Image0 (on), frames (on,on), planes (off,off,off,off,off,off), lut 0, x 0, y 0, z 1, Reduction
off )
      PauseText  ( “Ensure incident lighting is correct (WL = 0.88 − 0.94) and acquire image.” )
      Image Setup DC Twain [PAUSE] ( Camera 1, AutoExposure Off, Gain 0.00, ExposureTime 34.23
msec, Brightness 0, Lamp 38.83 )
      Acquire  ( into Image0 )
      DETECT - Projections only
      PauseText  ( “Ensure that threshold is set at least to the right of the left gray-level histogram peak
which corresponds to the ‘land’ region.” )
      Detect [PAUSE] ( whiter than 129, from Image0 into Binary0 delineated )
      BINARY IMAGE PROCESSING
      Binary Amend  ( Close from Binary0 to Binary1, cycles 10, operator Disc, edge erode on )
      Binary Identify  ( FillHoles from Binary1 to Binary1 )
      Binary Amend  ( Open from Binary1 to Binary2, cycles 20, operator Disc, edge erode on )
      Binary Amend  ( Close from Binary2 to Binary3, cycles 8, operator Disc, edge erode on )
      PauseText  ( “Toggle <control> and <b> keys to check projection detection and correct if
necessary.” )
      Binary Edit [PAUSE] ( Reject from Binary3 to Binary3, nib Fill, width 2 )
      Binary Logical  ( copy Binary3, inverted to Binary4 )
      IMAGE ACQUISITION 2 - % Open Area & Aperture Size
      Measure frame  ( x 74, y 110, Width 1249, Height 875 )
      Display  ( Image0 (on), frames (on,on), planes (off,off,off,off,off,off), lut 0, x 0, y 0, z 1, Reduction
off )
      PauseText  ( “Turn off incident light & ensure transmitted lighting is correct (WL = 0.95) and
acquire image.” )
      Image Setup DC Twain [PAUSE] ( Camera 1, AutoExposure Off, Gain 0.00, ExposureTime 34.23
msec, Brightness 0, Lamp 38.83 )
      Acquire  ( into Image0 )
      ACQFILE$ = “C:\Images\94054 - Nhan\”+TITLE$+“_”+STR$(FIELD)+“.jpg”
      Write image  ( from ACQOUTPUT into file ACQFILE$ )
      DETECT - Open areas only
      Detect  ( whiter than 127, from Image0 into Binary10 delineated )
      BINARY IMAGE PROCESSING
      Binary Amend  ( Close from Binary10 to Binary11, cycles 5, operator Disc, edge erode on )
      Binary Identify  ( FillHoles from Binary11 to Binary12 )
      Binary Amend  ( Open from Binary12 to Binary13, cycles 10, operator Disc, edge erode on )
      Binary Identify  ( EdgeFeat from Binary13 to Binary14 )
      PauseText  ( “Ensure apertures are detected accurately.” )
      Binary Edit [PAUSE] ( Reject from Binary14 to Binary14, nib Fill, width 2 )
      FIELD MEASUREMENTS - % Open Area, Bump Density & Spacing
          -- % open area
      MFLDIMAGE = 10
      Measure field  ( plane MFLDIMAGE, into FLDRESULTS(1), statistics into FLDSTATS(7,1) )
            Selected parameters: Area %
      Field Histogram #1  ( Y Param Number, X Param Area %, from 0. to 60., linear, 20 bins )
      PERCOPENAREA = FLDRESULTS(1)
        -- bump density & spacing
      MFLDIMAGE = 3
      Measure field  ( plane MFLDIMAGE, into FLDRESULTS(5), statistics into FLDSTATS(7,5) )
            Selected parameters:  Area, Intercept H, Intercept V, Area %,
            Count/Area
      BUMPDENSITY = FLDRESULTS(5)
      MNSPACE1 = (FRMAREA-FLDRESULTS(1)/(FLDRESULTS(2)+FLDRESULTS(3))/2
      Field Histogram #2  ( Y Param Number, X Param MNSPACE1, from 0. to 50., linear, 25 bins )
      File  ( FIELD, channel #1, 0 digits after “.” )
      File  ( PERCOPENAREA, channel #1, 1 digit after “.” )
      File  ( BUMPDENSITY, channel #1, 1 digit after “.” )
      File  (MNSPACE1, channel #1, 1 digit after “.” )
      File Line  ( channel #1 )
      FEATURE MEASUREMENTS - Aperture and bump sizes
        -- Bump Size
      Measure feature  ( plane Binary3, 8 ferets, minimum area: 24, grey image: Image0 )
            Selected parameters: Area, X FCP, Y FCP, EquivDiam
      Feature Histogram #1  ( Y Param Number, X Param Area, from 1. to 100., logarithmic, 20 bins )
      Feature Histogram #2  ( Y Param Number, X Param EquivDiam, from 1. to 100., logarithmic, 20
bins )
        -- Aperture Size
      Measure feature  ( plane Binary14, 8 ferets, minimum area: 24, grey image: Image0 )
            Selected parameters: Area, X FCP, Y FCP, Roundness, AspectRatio,
            EquivDiam
      Feature Histogram #3  ( Y Param Number, X Param Area, from 1. to 100., logarithmic, 20 bins )
      Feature Histogram #4  ( Y Param Number, X Param EquivDiam, from 1. to 100., logarithmic, 20
bins )
      Feature Histogram #5  ( Y Param Number, X Param Roundness, from 0.8999999762 to
2.900000095, linear, 20 bins )
      Feature Histogram #6  (Y Param Number, X Param AspectRatio, from 1. to 3., linear, 20 bins )
      Stage  ( Step, Wait until stopped + 1100 msecs )
    Next  ( FIELD )
  Next  ( SAMPLE )
  File Line  ( channel #1 )
  File Line  ( channel #1 )
  OUTPUT FEATURE HISTOGRAMS
  File  ( “Bump Size (area - sq. mm)”, channel #1 )
  File Line  ( channel #1 )
  File Feature Histogram Results  ( #1, differential, statistics, bin details, channel #1 )
  File Line  ( channel #1 )
  File Line  ( channel #1 )
  File  ( “Bump Size (ECD - mm)”, channel #1 )
  File Line  ( channel #1 )
  File Feature Histogram Results  ( #2, differential, statistics, bin details, channel #1 )
  File Line  ( channel #1 )
  File Line  ( channel #1 )
  File  ( “Aperture Size (area - sq. mm)”, channel #1 )
  File Line  ( channel #1 )
  File Feature Histogram Results  ( #3, differential, statistics, bin details, channel #1 )
  File Line  ( channel #1 )
  File Line  ( channel #1 )
  File  ( “Aperture Size (ECD - mm)”, channel #1 )
  File Line  ( channel #1 )
  File Feature Histogram Results  ( #4, differential, statistics, bin details, channel #1 )
  File Line  ( channel #1 )
  File Line  ( channel #1 )
  File  ( “Aperture Roundness”, channel #1 )
  File Line  ( channel #1 )
  File Feature Histogram Results  ( #5, differential, statistics, bin details, channel #1 )
  File Line  ( channel #1 )
  File Line  ( channel #1 )
  File  ( “Aperture Aspect Ratio”, channel #1 )
  File Line  ( channel #1 )
  File Feature Histogram Results  ( #6, differential, statistics, bin details, channel #1 )
  File Line  ( channel #1 )
File Line  ( channel #1 )
Close File  ( channel #1 )
END
```

The QUIPS algorithm is executed using the QWIN Pro software platform. The analyst is initially prompted to enter the specimen set information which is sent to the EXCEL file.

The analyst then enters an image file prefix name corresponding to the specimen identification. This will be used by the algorithm to save images acquired during the analysis to a specified file location. The analyst is next prompted by a live image set up window on the computer monitor screen to place a specimen onto the auto-stage. The sample should be laid flat and gentle force applied at edges of the sample to remove any macro-wrinkles that may be present. At this time, the Circline fluorescent light 712 can be on to assist in positioning the specimen. Next, the analyst is prompted to adjust the incident Circline fluorescent incident light 712 via the Variable Auto-transformer to a white level reading of approximately 0.9. The sub-stage transmitted light should either be turned off at this time or masked using a piece of light-blocking, black construction paper placed over the three (3) inch by three (3) inch opening 722.

The analyst is now prompted to ensure that the detection threshold is set to the proper level for detection of the nodes 222 using the Detection window which is displayed on the computer monitor screen. Typically, the threshold is set using the white mode at a point approximately near the middle of the 8-bit gray-level range (e.g., 127). If necessary, the threshold level can be adjusted up or down so that the resulting detected binary will optimally encompass the nodes 222 shown in the acquired image.

After the algorithm automatically performs several binary image processing steps on the detected binary of the nodes 222, the analyst will be given an opportunity to re-check node detection and correct any inaccuracies. The analyst can toggle both the ‘control’ and ‘b’ keys simultaneously to re-check node detection against the underlying acquired gray-scale image. If necessary, the analyst can select from a set of binary editing tools (e.g. draw, reject, etc.) to make any minor adjustments. If care is taken to ensure proper illumination and detection in the previously described steps, little or no correction at this point should be necessary.

Next, the analyst is prompted to turn off the incident Circline fluorescent light 712 and either turn on the sub-stage transmitted light or remove the light blocking mask. The sub-stage transmitted light is adjusted by the LED power controller to a white level reading of approximately 0.95. At this point, the image focus can be optimized for the apertured zone (such as first feature zone 220) of the material 200 including apertures 221.

The algorithm, after performing additional operations on the resulting separate binary images for apertures 221, will then prompt the analyst to re-check aperture 221 detection against the underlying gray-scale image. If necessary, the analyst can select from a set of binary editing tools (e.g. draw, reject, etc.) to make any minor adjustments.

The algorithm will then automatically perform measurements and output the data into a designated EXCEL spreadsheet file.

Following the transfer of data, the algorithm will direct the auto-stage to move to the next field-of-view and the process of turning on the incident, Circline fluorescent light 712 and blocking the transmitted sub-stage lighting will begin again. This process will repeat four times so that there will be five sets of data from five separate field-of-view images per single sampling replicate.

After completion of the analysis, the following measurement parameter data will be located in the EXCEL file after measurements and data transfer has occurred:
Percent Open Area
Node Density (No. per sq. metre)
Node Spacing (mm)
Node Size (One histogram for area in mm² and one histogram for equivalent circular diameter in mm)
Aperture Size (One histogram for area in mm² and one histogram for equivalent circular diameter in mm)
Aperture Roundness
Aperture Aspect Ratio

The final specimen mean spread value is usually based on an N=5 analysis from five, separate, specimen subsample replicates. A comparison of the percent open area, aperture 221 (aperture) size and other parameters acquired by the algorithm between different specimens can be performed using a Student's T analysis at the 90% confidence level.

### Compression Linearity Test

Compression Linearity is measured using the Kawabata Evaluation System KES model FB-3, again available from Kato Tech Company.

The instrument is designed to measure the compression properties of materials by compressing the sample between two plungers. To measure the compression properties, the top plunger is brought down on the sample at a constant rate until it reaches the maximum preset force. The displacement of the plunger is detected by a potentiometer. The amount of pressure taken to compress the sample (P, gf/cm²) vs. thickness (displacement) of the material (T, mm) is plotted on the computer screen. For all the materials in this study, the following instrument settings were used:

Sensitivity = 2 × 5
Gear (speed) = 1 mm/50 sec
Fm set = 5.0
Stroke select = Max 5 mm
Compression area = 2 cm²
Time lag = standard
Max compression force = 50 gf
The KES algorithm calculates the following compression characteristic values and displays them on a
computer screen:
Compression Linearity (LC).
5 measurements were taken on each sample.

### Determination of Residual Fecal Material Simulant Test

As noted above, such test method is described as a “Determination of Residual Fecal Material Simulant” test method in U.S. Pat. No. 9,480,609, titled “Absorbent Article”, the entirety of which is hereby incorporated by reference to the extent not contradictory herewith. In general, urine and BM simulant excretions from a silicone-composite mannequin (representative of a typical size NB-S2 baby) are collected within a biologically relevant sized and fitted diaper (sample) while the mannequin is in the accepted supine (sleep) position. The simulant may represent or model runny, breast-fed baby BM. Volume and flow rates for the urine and BM simulant excretions are provided below.

| | | | Wait Time | | | Wait Time |
|---|---|---|---|---|---|---|
| | Saline Insult | Saline Flow | From Saline | BM Insult | BM Flow | After BM |
| Size | Volume | Rate | to BM | Volume | Rate | Insult |
| NB | 40 mL | 4.1 mL/s | 30 sec. | 11 mL | 20 mL/s | 5 min. |
| 1 | 40 mL | 4.1 mL/s | 30 sec. | 11 mL | 20 mL/s | 5 min. |
| 2 | 45 mL | 4.1 mL/s | 30 sec. | 11 mL | 20 mL/s | 5 min. |

The BM simulant on the mannequin can be measured after the wait time to determine the amount of residual fecal material on the sample.

### REFERENCE CHARACTERS

10 Absorbent article
11 Chassis
12 Front waist region
14 Rear waist region
16 Crotch region
18 Longitudinal side edge
19 Body facing surface
20 Longitudinal side edge
21 Perforated zone
22 Front waist edge
23 Apertures
24 Rear waist edge
25 Perforated zone
26 Outer cover
27 Apertures
28 Bodyside liner
29 Longitudinal axis
30 Longitudinal direction
31 Lateral axis
32 Lateral direction
34 Absorbent body
36 Longitudinal edge
38 Longitudinal edge
40 First end edge
42 Second end edge
44 Absorbent assembly
45 Body facing surface
50 Containment flap
52 Containment flap
54 Elasticated waist member
56 Body facing surface
60 Leg elastic member
62 Leg elastic member
64 Base portion
64a Proximal end
64b Distal end
66 Projection portion
68 Flap elastic members
70 Active flap elastic region
71 Tack-down region
72 First longitudinal side edge
74 Second longitudinal side edge
84 Tack-down region
86 Elastic members
91 Back fasteners
92 Front fasteners
94 Stretch component
96 Nonwoven carrier or hook base
98 Fastening component
200 Nonwoven material
210 Fibrous web
212 First end portion
214 Second end portion
216 First side portion
218 Second side portion
220 First perforated zone
221 Apertures
222 Nodes
230 Second perforated zone
231 Apertures
240 First surface
241 Base plane
242 Second surface
250 Distal end portion
252 Cap (node)
254 Wall (node)
LAA Length of absorbent article
L1 Length of web
WAA Width of absorbent article
W1 Width of web
TT Transverse direction
H Height (nodes)
TW Thickness (node wall)
TC Thickness (node cap)

### EXAMPLE EMBODIMENTS

First example embodiment: A forming surface for fluid-entangled nonwoven material, comprising: a wall having a first surface and a second surface, the first surface positioned opposite the second surface on the wall, the wall defining a plurality of formation holes, each of the plurality of formation holes comprising a blind hole and at least one outlet passage, wherein each blind hole extends from a first end portion to a second end portion, the first end portion of each blind hole positioned at the first surface of the wall, each blind hole extending towards the second surface of the wall such that the second end portion of each blind hole is recessed within the wall, wherein each of the at least one outlet passage extends from a first end portion to a second end portion, the first end portion of each of the at least one outlet passage positioned at the second end portion of each blind hole, the second end portion of each of the at least one outlet passage positioned at the second surface of the wall, wherein, for each of the plurality of formation holes, an area of the blind hole at the first end portion of the blind hole is greater than a collective area of the at least one outlet passage at the first end portion of the at least one passage, and a ratio of the area of the blind hole at the first end portion of the blind hole to the collective area of the at least one outlet passage at the first end portion of the at least one passage is between 2.75:1 and 11:1.

Second example embodiment: The forming surface of the first example embodiment, wherein the wall is a cylindrical wall, and the plurality of formation holes are distributed on the cylindrical wall such that the plurality of formation holes are distributed circumferentially and axially on the cylindrical wall.

Third example embodiment: The forming surface of either the first example embodiment or the second example embodiment, wherein the first and second end portions of each blind hole are spaced apart along a central axis of each blind hole, and the first end portion of each of the at least one outlet passage is offset from the central axis of each blind hole.

Fourth example embodiment: The forming surface of any one of the first through third example embodiments, wherein each of the at least one outlet passage extends about parallel to the central axis of each blind hole.

Fifth example embodiment: The forming surface of any one of the first through fourth example embodiments, wherein the at least one outlet passage comprises a plurality of passages.

Sixth example embodiment: The forming surface of any one of the first through fifth example embodiments, wherein the central axis of each blind hole intersects the wall at the second end portion of each blind hole.

Seventh example embodiment: The forming surface of any one of the first through sixth example embodiments, wherein the wall defines a formation surface at the second end portion of each blind hole, and the formation surface has a conical or a semispherical shape.

Eighth example embodiment: The forming surface of any one of the first through seventh example embodiments, wherein the first and second end portions of each blind hole are spaced apart along a central axis of each blind hole, and a cross-sectional area of each blind hole in a plane perpendicular to the central axis decreases towards the second end portion of each blind hole.

Nineth example embodiment: The forming surface of the eighth example embodiment, wherein the cross-sectional area of each blind hole in the plane perpendicular to the central axis is substantially constant from the first end portion of each blind hole to at least a middle portion of each blind hole that is positioned equidistant between the first and second end portions of each blind hole.

Tenth example embodiment: The forming surface of any one of the first through nineth example embodiments, wherein the wall comprises a first wall section and a second wall section, each blind hole defined in the first wall section, each of the at least one outlet passage defined in the second wall section.

Eleventh example embodiment: The forming surface of any one of the first through tenth example embodiments, wherein the wall comprises a perforated body and a plurality of inserts, each of the plurality of inserts received within a respective perforation of the perforated body, each of the plurality of insert defining a respective one of the plurality of formation holes, the plurality of inserts formed from discrete pieces of material from the perforated body.

Twelfth example embodiment: A forming surface for fluid-entangled nonwoven material, comprising: a wall having a first surface and a second surface, the first surface positioned opposite the second surface on the wall, the wall defining a plurality of formation holes, each of the plurality of formation holes comprising a blind hole and at least one outlet passage, wherein each blind hole extends from a first end portion to a second end portion, the first end portion of each blind hole positioned at the first surface of the wall, each blind hole extending towards the second surface of the wall such that the second end portion of each blind hole is recessed within the wall, wherein each of the at least one outlet passage extends from a first end portion to a second end portion, the first end portion of each of the at least one outlet passage positioned at the second end portion of each blind hole, the second end portion of each of the at least one outlet passage positioned at the second surface of the wall, wherein the first and second end portions of each blind hole are spaced apart along a central axis of each blind hole, and the first end portion of each of the at least one outlet passage is offset from the central axis of each blind hole.

Thirteenth example embodiment: The forming surface of the twelfth example embodiment, wherein the wall is a cylindrical wall, and the plurality of formation holes are distributed on the cylindrical wall such that the plurality of formation holes are distributed circumferentially and axially on the cylindrical wall.

Fourteenth example embodiment: The forming surface of either the twelfth example embodiment or the thirteenth example embodiment, wherein each of the at least one outlet passage extends about parallel to the central axis of each blind hole.

Fifteenth example embodiment: The forming surface of any one of the twelfth through fourteenth example embodiments, wherein the central axis of each blind hole intersects the wall at the second end portion of each blind hole.

Sixteenth example embodiment: The forming surface of any one of the twelfth through fifteenth example embodiments, wherein the wall defines a formation surface at the second end portion of each blind hole, and the formation surface has a conical or a semispherical shape.

Seventeenth example embodiment: The forming surface of any one of the twelfth through sixteenth example embodiments, wherein a cross-sectional area of each blind hole in a plane perpendicular to the central axis decreases towards the second end portion of each blind hole.

Eighteenth example embodiment: The forming surface of any one of the twelfth through seventeenth example embodiments, wherein the cross-sectional area of each blind hole in the plane perpendicular to the central axis is substantially constant from the first end portion of each blind hole to at least a middle portion of each blind hole that is positioned equidistant between the first and second end portions of each blind hole.

Nineteenth example embodiment: A forming surface for fluid-entangled nonwoven material, comprising: a wall having a first surface and a second surface, the first surface positioned opposite the second surface on the wall, the wall defining a plurality of formation holes, each of the plurality of formation holes comprising a blind hole and at least one outlet passage, wherein each blind hole extends from a first end portion to a second end portion, the first end portion of each blind hole positioned at the first surface of the wall, each blind hole extending towards the second surface of the wall such that the second end portion of each blind hole is recessed within the wall and terminates prior to the second surface of the wall.

Twentieth example embodiment: The forming surface of the nineteenth example embodiment, wherein the wall comprises a connecting surface and a forming surface, the connecting surface extending inwardly into the blind hole from the first end portion of the blind hole, a cross-section of the connecting surface in a plane perpendicular to a central axis of the blind hole being substantially constant along the central axis, the formation surface positioned proximate the second end portion of the blind hole, a cross-section of the formation surface in the plane perpendicular to the central axis of the blind hole decreasing along the central axis towards the second end portion of the blind hole.

Twenty-First example embodiment: The forming surface of either the nineteenth example embodiment or the twentieth example embodiment, wherein the formation surface is semi-spherical or conical.

1. A forming surface for fluid-entangled nonwoven material, comprising:
a wall having a first surface and a second surface, the first surface positioned opposite the second surface on the wall, the wall defining a plurality of formation holes, each of the plurality of formation holes comprising a blind hole and at least one outlet passage,
wherein each blind hole extends from a first end portion to a second end portion, the first end portion of each blind hole positioned at the first surface of the wall, each blind hole extending towards the second surface of the wall such that the second end portion of each blind hole is recessed within the wall,
wherein each of the at least one outlet passage extends from a first end portion to a second end portion, the first end portion of each of the at least one outlet passage positioned at the second end portion of each blind hole, the second end portion of each of the at least one outlet passage positioned at the second surface of the wall,
wherein, for each of the plurality of formation holes, an area of the blind hole at the first end portion of the blind hole is greater than a collective area of the at least one outlet passage at the first end portion of the at least one passage, and a ratio of the area of the blind hole at the first end portion of the blind hole to the collective area of the at least one outlet passage at the first end portion of the at least one passage is between 2.75:1 and 11:1.

2. The forming surface of claim 1, wherein the wall is a cylindrical wall, and the plurality of formation holes are distributed on the cylindrical wall such that the plurality of formation holes are distributed circumferentially and axially on the cylindrical wall.

3. The forming surface of claim 1, wherein the first and second end portions of each blind hole are spaced apart along a central axis of each blind hole, and the first end portion of each of the at least one outlet passage is offset from the central axis of each blind hole.

4. The forming surface of claim 1, wherein each of the at least one outlet passage extends about parallel to a central axis of each blind hole.

5. The forming surface of claim 1, wherein the at least one outlet passage comprises a plurality of passages.

6. The forming surface of claim 1, wherein a central axis of each blind hole intersects the wall at the second end portion of each blind hole.

7. The forming surface of claim 1, wherein the wall defines a formation surface at the second end portion of each blind hole, and the formation surface has a conical or a semispherical shape.

8. The forming surface of claim 1, wherein the first and second end portions of each blind hole are spaced apart along a central axis of each blind hole, and a cross-sectional area of each blind hole in a plane perpendicular to the central axis decreases towards the second end portion of each blind hole.

9. The forming surface of claim 8, wherein the cross-sectional area of each blind hole in the plane perpendicular to the central axis is substantially constant from the first end portion of each blind hole to at least a middle portion of each blind hole that is positioned equidistant between the first and second end portions of each blind hole.

10. The forming surface of claim 1, wherein the wall comprises a first wall section and a second wall section, each blind hole defined in the first wall section, each of the at least one outlet passage defined in the second wall section.

11. The forming surface of claim 1, wherein the wall comprises a perforated body and a plurality of inserts, each of the plurality of inserts received within a respective perforation of the perforated body, each of the plurality of insert defining a respective one of the plurality of formation holes, the plurality of inserts formed from discrete pieces of material from the perforated body.

12. A forming surface for fluid-entangled nonwoven material, comprising:
a wall having a first surface and a second surface, the first surface positioned opposite the second surface on the wall, the wall defining a plurality of formation holes, each of the plurality of formation holes comprising a blind hole and at least one outlet passage,
wherein each blind hole extends from a first end portion to a second end portion, the first end portion of each blind hole positioned at the first surface of the wall, each blind hole extending towards the second surface of the wall such that the second end portion of each blind hole is recessed within the wall,
wherein each of the at least one outlet passage extends from a first end portion to a second end portion, the first end portion of each of the at least one outlet passage positioned at the second end portion of each blind hole, the second end portion of each of the at least one outlet passage positioned at the second surface of the wall, and
wherein the first and second end portions of each blind hole are spaced apart along a central axis of each blind hole, and the first end portion of each of the at least one outlet passage is offset from the central axis of each blind hole.

13. The forming surface of claim 12, wherein the wall is a cylindrical wall, and the plurality of formation holes are distributed on the cylindrical wall such that the plurality of formation holes are distributed circumferentially and axially on the cylindrical wall.

14. The forming surface of claim 12, wherein each of the at least one outlet passage extends about parallel to the central axis of each blind hole.

15. The forming surface of claim 12, wherein the central axis of each blind hole intersects the wall at the second end portion of each blind hole.

16. The forming surface of claim 12, wherein the wall defines a formation surface at the second end portion of each blind hole, and the formation surface has a conical or a semispherical shape.

17. The forming surface of claim 12, wherein a cross-sectional area of each blind hole in a plane perpendicular to the central axis decreases towards the second end portion of each blind hole.

18. The forming surface of claim 17, wherein the cross-sectional area of each blind hole in the plane perpendicular to the central axis is substantially constant from the first end portion of each blind hole to at least a middle portion of each blind hole that is positioned equidistant between the first and second end portions of each blind hole.

19. A forming surface for fluid-entangled nonwoven material, comprising:
a wall having a first surface and a second surface, the first surface positioned opposite the second surface on the wall, the wall defining a plurality of formation holes, each of the plurality of formation holes comprising a blind hole and at least one outlet passage, and
wherein each blind hole extends from a first end portion to a second end portion, the first end portion of each blind hole positioned at the first surface of the wall, each blind hole extending towards the second surface of the wall such that the second end portion of each blind hole is recessed within the wall and terminates prior to the second surface of the wall.

20. The forming surface of claim 19, wherein the wall comprises a connecting surface and a formation surface, the connecting surface extending inwardly into the blind hole from the first end portion of the blind hole, a cross-section of the connecting surface in a plane perpendicular to a central axis of the blind hole being substantially constant along the central axis, the formation surface positioned proximate the second end portion of the blind hole, a cross-section of the formation surface in the plane perpendicular to the central axis of the blind hole decreasing along the central axis towards the second end portion of the blind hole.

21. The forming surface of claim 20, wherein the formation surface is semi-spherical or conical.
